(19) United States
(12) Patent Application Publication
(10) Pub. No.: US 2006/0162020 A1
(43) Pub. Date: Jul. 20, 2006

(54) Promoters for the expression of genes in tagetes

(75) Inventors: Matt Sauer, Quedlinburg (DE); Christel Renate Schopfer, Quedlinburg (DE); Ralf Flachmann, Quedlinburg (DE); Karin Herbes, Quedlinburg (DE); Martin Klebsatten, Quedlinburg (DE); Thomas Luck, Neustadt (DE); Dirk Voeste, Limburgerhof (DE); Angela-Maria Pfeiffer, Birkenheide (DE)

Correspondence Address: CONNOLLY BOVE LODGE & HUTZ, LLP, P O BOX 2207, WILMINGTON, DE, 19899, US

(73) Assignee: SunGene GmbH, Gatersleben (DE)

(21) Appl. No.: 10/568,741

(22) PCT Filed: Jul. 31, 2004
(86) PCT No.: PCT/EP04/08624

§ 371 Date: Feb. 17, 2006

(30) Foreign Application Priority Data
Aug. 18, 2003 (EP) PCT EP 03 09102
Aug. 18, 2003 (EP) PCT 03 09107
Aug. 18, 2003 (EP) PCT 03 09105
Aug. 18, 2003 (EP) PCT 03 09106
Aug. 18, 2003 (EP) PCT 03 09109
Aug. 18, 2003 (EP) PCT 03 09101
Feb. 17, 2004 (DE) 10 2004 007 623.5

(51) Int. Cl. A01H 5/00; C12N 15/82; C12N 5/04; A01H 1/00

(52) U.S. Cl. 800/282; 435/419; 435/468; 800/323

(57) ABSTRACT

The present invention relates to the use of promoters for the expression, preferably for the flower-specific expression, of genes in plants of the genus Tagetes, to the genetically modified plants of the genus Tagetes, and to a process for the preparation of biosynthetic products by culturing the genetically modified plants.

The present invention relates to the use of promoters for the expression, preferably for the flower-specific expression, of genes in plants of the genus Tagetes, to the genetically modified plants of the genus Tagetes, and to a process for the preparation of biosynthetic products by culturing the genetically modified plants.

Various biosynthetic products, such as, for example, fine chemicals, such as, inter alia, amino acids, vitamins, carotenoids, but also proteins, are prepared in cells by means of natural metabolic processes und are used in many branches of industry, including the foodstuffs, feedstuffs, cosmetic, feed, food and pharmaceutical industries.

These substances, which together are described as fine chemicals/proteins, comprise, inter alia, organic acids, both proteinogenic and nonproteinogenic amino acids, nucleotides and nucleosides, lipids and fatty acids, diols, carbohydrates, aromatic compounds, vitamins, carotenoids and cofactors, and also proteins and enzymes. Their production on the large-scale in some cases takes place by means of biotechnological processes using microorganisms which were developed in order to produce and secrete large amounts of the in each case desired substance.

Carotenoids are synthesized de novo in bacteria, algae, fungi and plants. In recent years, it has increasingly been attempted also to utilize plants as production organisms for fine chemicals, in particular for vitamins and carotenoids.

A natural mixture of the carotenoids lutein and zeaxanthin is extracted, for example, from the flowers of marigold plants (Tagetes plants) as “oleoresin”. This oleoresin is used both as an ingredient of food supplements and in the feed sector.

Lycopene from tomatoes is likewise used as a food supplement, while phytoene is mainly used in the cosmetic sector.

Ketocarotenoids, that is carotenoids which comprise at least one keto group, such as, for example, astaxanthin, canthaxanthin, echinenone, 3-hydroxyechinenone, 3′-hydroxyechinenone, adonirubin and adonixanthin are natural antioxidants and pigments which are produced by some algae, plants and microorganisms as secondary metabolites.

On account of their color-imparting properties, the ketocarotenoids and in particular astaxanthin are used as pigmenting aids in animal nutrition, in particular in trout, salmon and shrimp farming.

An economical biotechnological process for the production of natural, biosynthetic products and in particular carotenoids is therefore of great importance.

WO 0032788 describes some carotenoid biosynthesis genes from plants of the genus Tagetes and discloses how genetically modified plants of the genus Tagetes could be produced in order to obtain various carotenoid profiles in the petals and thus to produce certain carotenoids selectively. To this end, it is necessary to overexpress some biosynthesis genes and to suppress others.

For the overexpression of the newly found carotenoid biosynthesis genes in plants of the genus Tagetes, WO 0032788 postulates the petal-specific promoter of the ketolase from Adonis vernalis.

On account of a large number of possible difficulties in the overexpression of certain genes, there is a constant need to make available further promoters which make possible expression of genes in plants of the genus Tagetes.

The invention was therefore based on the object of making available further promoters which make possible the expression of genes in plants of the genus Tagetes.

Accordingly, it has been found that the promoters selected from the group consisting of

A) EPSPS promoter

B) B gene promoter

C) PDS promoter and

D) CHRC promoter

are very highly suitable for the expression of genes in plants of the genus Tagetes, with the proviso that genes from plants of the genus Tagetes which are expressed in wild-type plants of the genus Tagetes by the respective promoter are excluded.

The invention therefore relates to the use of a promoter selected from the group consisting of

A) EPSPS promoter

B) B gene promoter

C) PDS promoter and

D) CHRC promoter

for the expression of genes in plants of the genus Tagetes, with the proviso that genes from plants of the genus Tagetes which are expressed in wild-type plants of the genus Tagetes by the respective promoter are excluded.

Benfey et al. (Plant Cell Volume 2, pp. 849-856) describe the EPSPS promoter from Petunia as a petal-specific promoter for the expression of genes in Petunia hybrida.

Ronen et al. (PNAS Volume 97, Number 20,11102-11107) describe the B-GENE promoter from tomato as a flower-specific promoter for the expression of genes in tomatoes.

Corona et al. (Plant Journal Volume 9, Number 4, pp. 505-512), Mann et al. (Nature Biotechnology Volume 18, pp. 888-892) and Rosati et al. (Plant Journal Volume 24, Number 3, 413-419) describe the PDS promoter from tomato as a fruit- and flower-specific promoter for the expression of genes in tomatoes and tobacco.

Vishnevetsky et al. (Plant Journal Volume 20 Number 4 pp. 423-431) describe the CHRC promoter from cucumber as a flower-specific promoter for the expression of genes in cucumber, and further plants such as, for example, carnation, sunflower, tobacco.

Furthermore, numerous flower-specific promoters from various organisms are known in the literature. In this context, it has surprisingly been found that many of these promoters do not lead to the expression, in particular do not lead to the flower-specific or petal-specific expression, of genes in plants of the genus Tagetes.

It was therefore surprising that the promoters selected from the group consisting of

A) EPSPS promoter

B) B gene promoter

C) PDS promoter and

D) CHRC promoter

are very highly suitable for the expression, in particular for the flower-specific and particularly preferably for the petal-specific expression, of genes in plants of the genus Tagetes.

A promoter is understood according to the invention as meaning a nucleic acid having expression activity, that is a nucleic acid which, in functional linkage with a nucleic acid to be expressed, also described as a gene below, regulates the expression, that is the transcription and the translation of this nucleic acid or this gene.

“Transcription” is understood according to the invention as meaning the process by which, starting from a DNA matrix, a complementary RNA molecule is prepared. Proteins such as RNA polymerase, “sigma factors” and transcriptional regulator proteins are involved in this process. The RNA synthesized is then used as a matrix in the translation process, which then leads to the biosynthetically active protein.

A “functional linkage” is understood in this connection as meaning, for example, the sequential arrangement of one of the promoters according to the invention and a nucleic acid sequence to be expressed and, if appropriate, further regulative elements such as, for example, a terminator in such a way that each of the regulative elements can fulfill its function in the expression of the nucleic acid sequence. To this end, a direct linkage in the chemical sense is not imperative. Genetic control sequences, such as, for example, enhancer sequences, can also exert their function on the target sequence from positions which are further removed or even from other DNA molecules. Arrangements are preferred in which the nucleic acid sequence to be expressed or the gene to be expressed is positioned behind (i.e. at the 3′-end) of the promoter sequence according to the invention, such that both sequences are bonded covalently to one another. Preferably, the distance between the promoter sequence and the nucleic acid sequence to be expressed is in this case lower than 200 base pairs, particularly preferably less than 100 base pairs, very particularly preferably less than 50 base pairs.

“Expression activity” is understood according to the invention as meaning the amount of protein formed in a certain time by the promoter, that is the expression rate.

“Specific expression activity” is understood according to the invention as meaning the amount of protein per promoter formed in a certain time by the promoter.

In the case of a “caused expression activity” or “caused expression rate” in relation to a gene in comparison with the wild-type, in comparison with the wild-type the formation of a protein is thus caused which thus was not present in the wild-type.

In the case of an “increased expression activity” or “increased expression rate” in relation to a gene in comparison with the wild-type, in comparison with the wild-type the amount of protein formed in a certain time is thus increased.

The formation rate at which a biosynthetically active protein is prepared is a product of the rate of transcription and translation. Both rates can be influenced according to the invention and thus influence the rate of formation of products in a microorganism.

The indication “that genes from plants of the genus Tagetes which are expressed in wild-type plants of the of the genus Tagetes by the “respective” promoter are excluded” means that, for example, the EPSPS promoter from plants of the genus Tagetes is not used for the expression of EPSPS genes from plants of the genus Tagetes. On the other hand, the EPSPS gene from plants of the genus Tagetes can be expressed according to the invention by a B gene promoter, PDS promoter or CHRC promoter from plants of the genus Tagetes.

The term “wild-type” or “wild-type plant” is understood according to the invention as meaning the corresponding starting plant of the genus Tagetes.

Depending on the connection, the term “plant” can be understood as meaning the starting plant (wild-type) or a genetically modified plant according to the invention of the genus Tagetes or both.

Preferably, “wild-type” is understood as meaning the plant Tagetes erecta, in particular the plant Tagetes erecta Hybrid 50011 (WO 02012438) as a reference organism for the increasing or causing of the expression activity or expression rate and for the increasing of the content of biosynthetic products.

An “EPSPS promoter” is understood as meaning promoters which naturally regulate the gene expression of a nucleic acid encoding a 5-enolpyruvyl shikimate 3-phosphate synthase in organisms, preferably in plants, and nucleic acid sequences derivable from these promoter sequences by substitution, insertion or deletion of nucleotides or by fragmentation of these promoter sequences, and which additionally have this expression activity and thus represent functional equivalents.

These EPSPS promoter sequences from other organisms, in particular plants, as the promoter sequences indicated below, can be found, in particular, by homology comparisons in databases or hybridization studies with DNA libraries of various organisms using the EPSPS promoter sequences or the nucleic acids, encoding a 5-enolpyruvyl shikimate 3-phosphate synthase, described below.

Preferably, to this end the nucleic acids encoding a 5-enolpyruvyl shikimate 3phosphate synthase are used, as in the encoding sequence conserved regions are more frequent than in the promoter sequence.

A 5-enolpyruvyl shikimate 3-phosphate synthase is understood as meaning a protein which has the enzymatic activity to convert shikimate 3-phosphate to 5-enolpyruvyl shikimate 3-phosphate.

Preferred EPSPS promoters comprise
A1) the nucleic acid sequence SEQ. ID. NO. 1, 2 or 3 or
A2) a sequence derived from these sequences by substitution, insertion or deletion of nucleotides, which has an identity of at least 60% at nucleic acid level with the respective sequence SEQ. ID. NO. 1, 2 or 3 or
A3) a nucleic acid sequence which is hybridized with the nucleic acid sequence SEQ. ID. NO. 1, 2 or 3 under stringent conditions or
A4) functionally equivalent fragments of the sequences under A1), A2) or A3)

The nucleic acid sequence SEQ. ID. NO. 1 represents a promoter sequence of the 5-enolpyruvyl shikimate 3-phosphate synthase (EPSPS) from Petunia hybrida (AAH 19653).

The nucleic acid sequence SEQ. ID. NO. 2 represents a promoter sequence of the 5-enolpyruvyl shikimate 3-phosphate synthase (EPSPS) from Petunia hybrida (M37029).

The nucleic acid sequence SEQ. ID. NO. 3 represents a further promoter sequence of the 5-enolpyruvyl shikimate 3-phosphate synthase (EPSPS) from Petunia hybrida.

The invention furthermore relates to EPSPS promoters, comprising a sequence derived from these sequences (SEQ. ID. NO. 1, 2 or 3) by substitution, insertion or deletion of nucleotides, which has an identity of at least 60% at nucleic acid level with the respective sequence SEQ. ID. NO. 1, 2 or 3.

Further natural examples according to the invention of EPSPS promoters according to the invention can easily be found, for example, from various organisms whose genomic sequence is known, by identity comparisons of the nucleic acid sequences from databases containing the sequences SEQ ID NO: 1, 2 or 3 described above.

Synthetic EPSPS promoter sequences according to the invention can easily be found starting from the sequences SEQ ID NO: 1, 2 or 3 by synthetic variation and mutation, for example by substitution, insertion or deletion of nucleotides.

The following definitions and conditions of the identity comparisons and hybridization conditions apply for all nucleic acids, that is all promoters and genes of the description.

The term “substitution” is to be understood as meaning the exchange of one or more nucleotides for one or more nucleotides. “Deletion” is the replacement of a nucleotide by a direct bond. Insertions are insertions of nucleotides into the nucleic acid sequence, a direct bond formally being replaced by one or more nucleotides.

Identity between two nucleic acids is understood as meaning the identity of the nucleotides over the entire nucleic acid length in each case, in particular the identity which is calculated by comparison with the aid of the Vector NTI Suite 7.1 software of Informax (USA) using the Clustal method (Higgins D G, Sharp P M. Fast and sensitive multiple sequence alignments on a microcomputer. Comput Appl. Biosci. 1989 April; 5(2):151-1) with setting of the following parameters:

Multiple alignment parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise alignment parameter:

| FAST algorithm | on |
|---|---|
| K-tuple size | 1 |
| Gap penalty | 3 |
| Window size | 5 |
| Number of best diagonals | 5 |

A nucleic acid sequence which has an identity of at least 60% with the sequence SEQ ID NO: 1 is accordingly understood as meaning a nucleic acid sequence which, in a comparison of its sequence with the sequence SEQ ID NO: 1, in particular according to the above program logarithm, has an identity of at least 60% with the above parameter set.

A nucleic acid sequence which has an identity of at least 60% with the sequence SEQ ID NO: 2 is accordingly understood as meaning a nucleic acid sequence which, in a comparison of its sequence with the sequence SEQ ID NO: 2, in particular according to the above program logarithm, has an identity of at least 60% with the above parameter set.

A nucleic acid sequence which has an identity of at least 60% with the sequence SEQ ID NO: 3 is accordingly understood as meaning a nucleic acid sequence which, in a comparison of its sequence with the sequence SEQ ID NO: 3, in particular according to the above program logarithm, has an identity of at least 60% with the above parameter set.

Particularly preferred EPSPS promoters have, with the respective nucleic acid sequence SEQ. ID. NO. 1, 2 or 3, an identity of at least 70%, more preferably at least 80%, at least 90%, at least 92%, at least 95%, at least 96%, at least 97%, at least 98%, particularly preferably at least 99%.

Further natural examples of EPSPS promoters can furthermore easily be found starting from the nucleic acid sequences described above, in particular starting from the sequences SEQ ID NO: 1, 2 or 3, of various organisms whose genomic sequence is not known, by hybridization techniques in a manner known per se.

A further subject of the invention therefore relates to EPSPS promoters comprising a nucleic acid sequence which hybridizes with the nucleic acid sequence SEQ. ID. No. 1, 2 or 3 under stringent conditions. This nucleic acid sequence comprises at least 10, more preferably more than 12, 15, 30, 50 or particularly preferably more than 150 nucleotides.

“Hybridization” is understood as meaning the ability of a poly- or oligonucleotide to bind under stringent conditions to a nearly complementary sequence, while under these conditions nonspecific binding between noncomplementary partners does not happen. To this end, the sequences should preferably be complementary to 90-100%. The characteristic of complementary sequences to be able to bind specifically to one another is made use of, for example, in the Northern or Southern blot technique or in primer binding in PCR or RT-PCR.

Hybridization takes place according to the invention under stringent conditions. Such hybridization conditions are described, for example, in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6:

Stringent hybridization conditions are in particular understood as meaning: Overnight incubation at 42° C. in a solution consisting of 50% formamide, 5×SSC (750 mM NaCl, 75 mM trisodium citrate), 50 mM sodium phosphate (ph 7.6), 5× Denhardt solution, 10% dextran sulfate and 20 g/ml of denatured, sheared salmon sperm DNA, followed by washing of the filter with 0.1×SSC at 65° C.

For promoters, a “functionally equivalent fragment” is understood as meaning fragments essentially having the same promoter activity as the starting sequence.

“Essentially identical” is understood as meaning a specific expression activity which has at least 50%, preferably 60%, more preferably 70%, more preferably 80%, more preferably 90%, particularly preferably 95%, of the specific expression activity of the starting sequence.

“Fragments” are understood as meaning subsequences of the EPSPS promoters described by embodiment A1), A2) or A3). Preferably, these fragments have more than 10, but more preferably more than 12, 15, 30, 50 or particularly preferably more than 150, connected nucleotides of the nucleic acid sequence SEQ. ID. NO. 1, 2 or 3.

The use of the nucleic acid sequence SEQ. ID. NO. 1, 2 or 3 as an EPSPS promoter, i.e. for the expression of genes in plants of the genus Tagetes, is particularly preferred.

All abovementioned EPSPS promoters can furthermore be prepared in a manner known per se by chemical synthesis from the nucleotide structural units such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pp. 896-897). The addition of synthetic oligonucleotides and filling of gaps with the aid of the Klenow fragment of the DNA polymerase and ligation reactions and general cloning processes are described in Sambrook et al. (1989), Molecular cloning: A laboratory manual, Cold Spring Harbor Laboratory Press.

A “B gene promoter” is understood as meaning promoters which naturally regulate the gene expression of a nucleic acid encoding a lycopene β-cyclase in organisms, preferably in plants, in particular a chromoplast-specific lycopene β-cyclase, and nucleic acid sequences derivable from these promoter sequences by substitution, insertion or deletion of nucleotides or by fragmentation of these promoter sequences, and which additionally have this expression activity and thus represent functional equivalents.

These B gene promoter sequences from other organisms, in particular plants, as the promoter sequences indicated below, can in particular be found by homology comparisons in databases or hybridization studies with DNA libraries of various organisms using the B gene promoter sequences described below or the nucleic acids encoding a lycopene β-cyclase.

Preferably, to this end the nucleic acids encoding a lycopene β-cyclase are used, since in the coding sequence conserved regions are more frequent than in the promoter sequence.

A lycopene β-cyclase is understood as meaning a protein which has the enzymatic activity to convert lycopene to γ-carotene and/or β-carotene.

Preferred B gene promoters comprise
B1) the nucleic acid sequence SEQ. ID. NO. 4, 5 or 6 or
B2) a sequence derived from these sequences by substitution, insertion or deletion of nucleotides, which has an identity of at least 60% at nucleic acid level with the respective sequence SEQ. ID. NO. 4, 5 or 6 or
B3) a nucleic acid sequence which hybridizes with the nucleic acid sequence SEQ. ID. NO. 4, 5 or 6 under stringent conditions or
B4) functionally equivalent fragments of the sequences under B1), B2) or B3).

The nucleic acid sequence SEQ. ID. NO. 4 represents a promoter sequence of the chromoplast-specific lycopene β-cyclase (B gene) from Lycopersicon esculentum (AAZ51517).

The nucleic acid sequence SEQ. ID. NO. 5 represents a promoter sequence of the chromoplast-specific lycopene β-cyclase (B gene) from Lycopersicon esculentum (AAZ51521).

The nucleic acid sequence SEQ. ID. NO. 6 represents a further promoter sequence of the chromoplast-specific lycopene β-cyclase (B gene) from Lycopersicon esculentum.

The invention furthermore relates to B gene promoters comprising a sequence derived from these sequences (SEQ. ID. NO. 4, 5 or 6) by substitution, insertion or deletion of nucleotides, which has an identity of at least 60% at nucleic acid level with the respective sequence SEQ. ID. NO. 4, 5 or 6.

Further natural examples according to the invention of B gene promoters according to the invention can easily be found, for example, from various organisms whose genomic sequence is known, by identity comparisons of the nucleic acid sequences from databases with the sequences SEQ ID NO: 4, 5 or 6 described above.

Synthetic B gene promoter sequences according to the invention can easily be found starting from the sequences SEQ ID NO: 4, 5 or 6 by synthetic variation and mutation, for example by substitution, insertion or deletion of nucleotides.

A nucleic acid sequence which has an identity of at least 60% with the sequence SEQ ID NO: 4 is accordingly understood as meaning a nucleic acid sequence which, in a comparison of its sequence with the sequence SEQ ID NO: 4, in particular according to the above program logarithm with the above parameter set, has an identity of at least 60%.

A nucleic acid sequence which has an identity of at least 60% with the sequence SEQ ID NO: 5 is accordingly understood as meaning a nucleic acid sequence which, in a comparison of its sequence with the sequence SEQ ID NO: 5, in particular according to the above program logarithm with the above parameter set, has an identity of at least 60%.

A nucleic acid sequence which has an identity of at least 60% with the sequence SEQ ID NO: 6 is accordingly understood as meaning a nucleic acid sequence which, in a comparison of its sequence with the sequence SEQ ID NO: 6, in particular according to the above program logarithm with the above parameter set, has an identity of at least 60%.

Particularly preferred B gene promoters have, with the respective nucleic acid sequence SEQ. ID. NO. 4, 5 or 6, an identity of at least 70%, more preferably at least 80%, at least 90%, at least 92%, at least 95%, at least 96%, at least 97%, at least 98%, particularly preferably at least 99%.

Further natural examples of B gene promoters can furthermore be easily found starting from the nucleic acid sequences described above, in particular starting from the sequences SEQ ID NO: 4, 5 or 6 from various organisms whose genomic sequence is not known, by hybridization techniques in a manner known per se.

A further subject of the invention therefore relates to B gene promoters, comprising a nucleic acid sequence which hybridizes with the nucleic acid sequence SEQ. ID. No. 4, 5 or 6 under stringent conditions. This nucleic acid sequence comprises at least 10, more preferably more than 12, 15, 30, 50 or particularly preferably more than 150 nucleotides.

The hybridization conditions are described above.

For promoters, a “functionally equivalent fragment” is understood as meaning fragments which essentially have the same promoter activity as the starting sequence.

“Essentially identical” is understood as meaning a specific expression activity which has at least 50%, preferably 60%, more preferably 70%, more preferably 80%, more preferably 90%, particularly preferably 95%, of the specific expression activity of the starting sequence.

“Fragments” are understood as meaning subsequences of the B gene promoters described by embodiment B1), B2) or B3). Preferably, these fragments have more than 10, but more preferably more than 12, 15, 30, 50 or particularly preferably more than 150, connected nucleotides of the nucleic acid sequence SEQ. ID. NO. 4, 5 or 6.

The use of the nucleic acid sequence SEQ. ID. NO. 4, 5 or 6 as a B gene promoter, i.e. for the expression of genes in plants of the genus Tagetes, is particularly preferred.

All abovementioned B gene promoters can furthermore be prepared in a manner known per se by chemical synthesis from the nucleotide structural units such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pp. 896-897). The addition of synthetic oligonucleotides and filling of gaps with the aid of the Klenow fragment of the DNA polymerase and ligation reactions and general cloning processes are described in Sambrook et al. (1989), Molecular cloning: A laboratory manual, Cold Spring Harbor Laboratory Press.

A “PDS promoter” is understood as meaning promoters which, in organisms, preferably in plants, naturally regulate the gene expression of a nucleic acid encoding a phytoene desaturase, and nucleic acid sequences derivable from these promoter sequences by substitution, insertion or deletion of nucleotides or by fragmentation of these promoter sequences, and which additionally have this expression activity and thus represent functional equivalents.

These PDS promoter sequences from other organisms, in particular plants, as the promoter sequences indicated below, can be found, in particular, by homology comparisons in databases or hybridization studies with the DNA libraries of various organisms using the PDS promoter sequences described below or the nucleic acids encoding a phytoene desaturase.

Preferably, to this end the nucleic acids encoding a phytoene desaturase are used, as conserved regions are more frequent in the coding sequence than in the promoter sequence.

A phytoene desaturase is preferably understood as meaning a protein which has the enzymatic activity to convert phytoene to phytofluene.

Preferred PDS promoters comprise
C1) the nucleic acid sequence SEQ. ID. NO. 7, 8, 9 or 10 or
C2) a sequence derived from these sequences by substitution, insertion or deletion of nucleotides, which has an identity of at least 60% at nucleic acid level with the respective sequence SEQ. ID. NO. 7, 8, 9 or 10 or
C3) a nucleic acid sequence which hybridizes with the nucleic acid sequence SEQ. ID. NO. 7, 8, 9 or 10 under stringent conditions or
C4) functionally equivalent fragments of the sequences under C1), C2) or C3).

The nucleic acid sequence SEQ. ID. NO. 7 represents a promoter sequence of the phytoene desaturase (PDS) from Lycopersicon esculentum (U46919).

The nucleic acid sequence SEQ. ID. NO. 8 represents a promoter sequence of the phytoene desaturase (PDS) from Lycopersicon esculentum (X78271).

The nucleic acid sequence SEQ. ID. NO. 9 represents a promoter sequence of the phytoene desaturase (PDS) from Lycopersicon esculentum (X171023).

The nucleic acid sequence SEQ. ID. NO. 10 represents a further promoter sequence of the phytoene desaturase (PDS) from Lycopersicon esculentum.

The invention furthermore relates to PDS promoters comprising a sequence derived from these sequences (SEQ. ID. NO. 7, 8, 9 or 10) by substitution, insertion or deletion of nucleotides, which has an identity of at least 60% at nucleic acid level with the respective sequence SEQ. ID. NO. 7, 8, 9 or 10.

Further natural examples according to the invention of PDS promoters according to the invention can easily be found, for example, from various organisms whose genomic sequence is known, by identity comparisons of the nucleic acid sequences from databases with the sequences SEQ ID NO: 7, 8, 9 or 10 described above.

Synthetic PDS promoter sequences according to the invention can easily be found starting from the sequences SEQ ID NO: 7, 8, 9 or 10 by synthetic variation and mutation, for example by substitution, insertion or deletion of nucleotides.

A nucleic acid sequence which has an identity of at least 60% with the sequence SEQ ID NO: 7 is accordingly understood as meaning a nucleic acid sequence which, in a comparison of its sequence with the sequence SEQ ID NO: 7, in particular according to the above program logarithm, has an identity of at least 60% with the above parameter set.

A nucleic acid sequence which has an identity of at least 60% with the sequence SEQ ID NO: 8 is accordingly understood as meaning a nucleic acid sequence which, in a comparison of its sequence with the sequence SEQ ID NO: 8, in particular according to the above program logarithm, has an identity of at least 60% with the above parameter set.

A nucleic acid sequence which has an identity of at least 60% with the sequence SEQ ID NO: 9 is accordingly understood as meaning a nucleic acid sequence which, in a comparison of its sequence with the sequence SEQ ID NO: 9, in particular according to the above program logarithm, has an identity of at least 60% with the above parameter set.

A nucleic acid sequence which has an identity of at least 60% with the sequence SEQ ID NO: 10 is accordingly understood as meaning a nucleic acid sequence which, in a comparison of its sequence with the sequence SEQ ID NO: 10, in particular according to the above program logarithm, has an identity of at least 60% with the above parameter set.

Particularly preferred PDS promoters have an identity of at least 70%, more preferably at least 80%, at least 90%, at least 92%, at least 95%, at least 96%, at least 97%, at least 98%, particularly preferably at least 99%, with the respective nucleic acid sequence SEQ. ID. NO. 7, 8, 9 or 10.

Further natural examples of PDS promoters can furthermore easily be found by hybridization techniques in a manner known per se starting from the nucleic acid sequences described above, in particular starting from the sequences SEQ ID NO: 7, 8, 9 or 10 of various organisms whose genomic sequence is not known.

A further subject of the invention therefore relates to PDS promoters comprising a nucleic acid sequence which hybridizes with the nucleic acid sequence SEQ. ID. No. 7, 8, 9 or 10 under stringent conditions. This nucleic acid sequence comprises at least 10, more preferably more than 12, 15, 30, 50 or particularly preferably more than 150, nucleotides.

The hybridization conditions are described above.

For promoters, a “functionally equivalent fragment” is understood as meaning fragments which have essentially the same promoter activity as the starting sequence.

“Essentially identical” is understood as meaning a specific expression activity which has at least 50%, preferably 60%, more preferably 70%, more preferably 80%, more preferably 90%, particularly preferably 95%, of the specific expression activity of the starting sequence.

“Fragments” are understood as meaning subsequences of the PDS promoters described by embodiment C1), C2) or C3). Preferably, these fragments have more than 10, but more preferably more than 12, 15, 30, 50 or particularly preferably more than 150, connected nucleotides of the nucleic acid sequence SEQ. ID. NO. 7, 8, 9 or 10.

The use of the nucleic acid sequence SEQ. ID. NO. 7, 8, 9 or 10 as a PDS promoter, i.e. for the expression of genes in plants of the genus Tagetes, is particularly preferred.

All abovementioned PDS promoters can furthermore be prepared in a manner known per se by chemical synthesis from the nucleotide structural units, such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pp. 896-897). The addition of synthetic oligonucleotides and filling of gaps with the aid of the Klenow fragment of the DNA polymerase and ligation reactions and general cloning processes are described in Sambrook et al. (1989), Molecular cloning: A laboratory manual, Cold Spring Harbor Laboratory Press.

A “CHRC promoter” is understood as meaning promoters which naturally regulate the gene expression of a nucleic acid encoding a chromoplast-associated protein C in organisms, preferably in plants, and nucleic acid sequences derivable from these promoter sequences by substitution, insertion or deletion of nucleotides or by fragmentation of these promoter sequences, which additionally have this expression activity and thus represent functional equivalents.

These CHRC promoter sequences from other organisms, in particular plants, as the promoter sequences indicated below, can in particular be found by homology comparisons in databases or hybridization studies with DNA libraries of various organisms using the CHRC promoter sequences described below or the nucleic acids encoding a chromoplast-associated protein C.

Preferably, for this the nucleic acids encoding a chromoplast-associated protein C are used, as in the coding sequence conserved regions are more frequent than in the promoter sequence.

Preferred CHRC promoters comprise
D1) the nucleic acid sequence SEQ. ID. NO. 11, 12, 13 or 14 or
D2) a sequence derived from these sequences by substitution, insertion or deletion of nucleotides, which has an identity of at least 60% at nucleic acid level with the respective sequence SEQ. ID. NO. 11, 12, 13 or 14 or
D3) a nucleic acid sequence which hybridizes with the nucleic acid sequence SEQ. ID. NO. 11, 12, 13 or 14 under stringent conditions or
D4) functionally equivalent fragments of the sequences under D1), D2) or D3).

The nucleic acid sequence SEQ. ID. NO. 11 represents a promoter sequence of the chromoplast-associated protein C(CHRC) from cucumber (AAV36416).

The nucleic acid sequence SEQ. ID. NO. 12 represents a further promoter sequence of the chromoplast-associated protein C(CHRC) from cucumber.

The nucleic acid sequence SEQ. ID. NO. 13 represents a further promoter sequence of the chromoplast-associated protein C(CHRC) from cucumber.

The nucleic acid sequence SEQ. ID. NO. 14 represents a further promoter sequence of the chromoplast-associated protein C(CHRC) from cucumber.

The invention furthermore relates to CHRC promoters comprising a sequence derived from these sequences (SEQ. ID. NO. 11, 12, 13, or 14) by substitution, insertion or deletion of nucleotides, which has an identity of at least 60% at nucleic acid level with the respective sequence SEQ. ID. NO. 11, 12, 13, or 14.

Further natural examples according to the invention of CHRC promoters according to the invention can, for example, easily be found from various organisms whose genomic sequence is known, by identity comparisons of the nucleic acid sequences from databases with the sequences SEQ ID NO: 11, 12, 13, or 14 described above.

Synthetic CHRC promoter sequences according to the invention can easily be found starting from the sequences SEQ ID NO: 11, 12, 13, or 14 by synthetic variation and mutation, for example by substitution, insertion or deletion of nucleotides.

A nucleic acid sequence which has an identity of at least 60% with the sequence SEQ ID NO: 11 is accordingly understood as meaning a nucleic acid sequence which, in a comparison of its sequence with the sequence SEQ ID NO: 11, in particular according to the above program logarithm, has an identity of at least 60% with the above parameter set.

A nucleic acid sequence which has an identity of at least 60% with the sequence SEQ ID NO: 12 is accordingly understood as meaning a nucleic acid sequence which, in a comparison of its sequence with the sequence SEQ ID NO: 12, in particular according to the above program logarithm, has an identity of at least 60% with the above parameter set.

A nucleic acid sequence which has an identity of at least 60% with the sequence SEQ ID NO: 13 is accordingly understood as meaning a nucleic acid sequence which, in a comparison of its sequence with the sequence SEQ ID NO: 13, in particular according to the above program logarithm, has an identity of at least 60% with the above parameter set.

A nucleic acid sequence which has an identity of at least 60% with the sequence SEQ ID NO: 14 is accordingly understood as meaning a nucleic acid sequence which, in a comparison of its sequence with the sequence SEQ ID NO: 14, in particular according to the above program logarithm, has an identity of at least 60% with the above parameter set.

Particularly preferred CHRC promoters have, with the respective nucleic acid sequence SEQ. ID. NO. 11, 12, 13, or 14, an identity of at least 70%, more preferably at least 80%, at least 90%, at least 92%, at least 95%, at least 96%, at least 97%, at least 98%, particularly preferably at least 99%.

Further natural examples of CHRC promoters can furthermore easily be found starting from the nucleic acid sequences described above, in particular starting from the sequences SEQ ID NO: 11, 12, 13, or 14 of various organisms whose genomic sequence is not known, by hybridization techniques in a manner known per se.

A further subject of the invention therefore relates to CHCRC promoters comprising a nucleic acid sequence, which hybridizes with the nucleic acid sequence SEQ. ID. No. 11, 12, 13, or 14 under stringent conditions. This nucleic acid sequence comprises at least 10, more preferably more than 12, 15, 30, 50 or particularly preferably more than 150 nucleotides.

The hybridization conditions are described above.

For promoters, a “functionally equivalent fragment” is understood as meaning fragments which essentially have the same promoter activity as the starting sequence.

“Essentially identical” is understood as meaning a specific expression activity which has at least 50%, preferably 60%, more preferably 70%, more preferably 80%, more preferably 90%, particularly preferably 95%, of the specific expression activity of the starting sequence.

“Fragments” are understood as meaning subsequences of the CHRC promoters described by embodiment D1), D2) or D3). Preferably, these fragments have more than 10, more preferably, however, more than 12, 15, 30, 50 or particularly preferably more than 150, connected nucleotides of the nucleic acid sequence SEQ. ID. NO. 11, 12, 13, or 14.

The use of the nucleic acid sequence SEQ. ID. NO. 11, 12, 13, or 14 as a CHRC promoter, i.e. for the expression of genes in plants of the genus Tagetes, is particularly preferred.

All abovementioned CHRC promoters can furthermore be prepared in a manner known per se by chemical synthesis from the nucleotide structural units, such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pp. 896-897). The addition of synthetic oligonucleotides and filling of gaps with the aid of the Klenow fragment of the DNA polymerase and ligation reactions and general cloning processes are described in Sambrook et al. (1989), Molecular cloning: A laboratory manual, Cold Spring Harbor Laboratory Press.

Using the promoters according to the invention, in principle any gene, that is any nucleic acid, encoding a protein, can be expressed in plants of the genus Tagetes, in particular expressed flower-specifically, particularly preferably expressed petal-specifically.

These genes to be expressed in plants of the genus Tagetes are also called “effect genes” below.

Preferred effect genes are, for example, genes from the biosynthesis pathway of odoriferous substances and flower colors whose expression or increased expression in plants of the genus Tagetes leads to a change in the odor and/or the flower color of flowers of the plants of the genus Tagetes.

The biosynthesis of volatile odor components, especially in flowers, was in recent years studied on various model organisms such as Clarkia breweri and Antirhinum majus L. Volatile odor components are formed, for example, within the monoterpene and phenylpropane metabolism. In the first case, the component is linalool; methyleneeugenol, benzyl acetate, methyl benzoate and methyl salicate are derived from the phenylpropanes.

For the biosynthesis of linalool, (ISo)methyleigenol, benzyl acetate and methyl salicinate, preferred genes are selected from the group consisting of nucleic acids encoding a linalool synthase (LIS), nucleic acids encoding an S-adenosyl-L-Met:(iso)-eugenol O-methyltransferase (IEMT), nucleic acids encoding an acetyl-CoA-benzyl alcohol acetyltransferase and nucleic acids encoding an S-adenosyl-L-Met: salicylic acid methyltransferase (SAMT). Nucleic acid sequences and protein sequences for the enzymatic activities mentioned are described in Dudareva et al. Plant Cell 8 (1996), 1137-1148; Wang et al. Plant Physiol. 114 (1997), 213-221 and Dudareva et al. Plant J. 14 (1998) 297-304.

Particularly preferred effect genes are genes from biosynthesis pathways of biosynthetic products which can be prepared in plants of the genus Tagetes naturally, i.e. in the wild-type or by genetic modification of the wild-type, in particular can be prepared in flowers, particularly preferably can be prepared in petals.

Preferred biosynthetic products are fine chemicals.

The term “fine chemical” is known in the specialist field and comprises compounds which are produced by an organism and are applied in various branches of industry, such as, for example, but not restricted to, the pharmaceutical industry, the agricultural industry, cosmetics, food and feed industries. These compounds comprise organic acids, such as, for example, tartaric acid, itaconic acid and diaminopimelic acid, both proteinogenic and nonproteinogenic amino acids, purine and pyrimidine bases, nucleosides and nucleotides (as described, for example, in Kuninaka, A. (1996) Nucleotides and related compounds, pp. 561-612, in Biotechnology vol. 6, Rehm et al., publ. VCH: Weinheim and the citation contained therein), lipids, saturated and unsaturated fatty acids (for example arachidonic acid), diols (for example propanediol and butanediol), carbohydrates (for example hyaluronic acid and trehalose), aromatic compounds (for example aromatic amines, vanillin and indigo), vitamins, carotenoids and cofactors (as described in Ullmann's Encyclopedia of Industrial Chemistry, vol. A27, “Vitamins”, pp. 443-613 (1996) VCH: Weinheim and the citation contained therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held on Sep. 1st-3rd, 1994 in Penang, Malysia, AOCS Press (1995)), enzymes and all other chemicals described by Gutcho (1983) in Chemicals by Fermentation, Noyes Data Corporation, ISBN: 0818805086 and the references stated therein. The metabolism and the uses of certain fine chemicals are further illustrated below.

I. Amino Acid Metabolism and Uses

The amino acids comprise the fundamental structural units of all proteins and are thus essential for the normal cell functions. The term “amino acid” is known in the specialist field. The proteinogenic amino acids, of which there are 20 types, serve as structural units for proteins, in which they are linked to one another by means of peptide bonds, whereas the nonproteinogenic amino acids (of which hundreds are known) usually do not occur in proteins (see Ullmann's Encyclopedia of Industrial Chemistry, vol. A2, pp. 57-97 VCH: Weinheim (1985)). The amino acids can be present in the D- or L-configuration, although L-amino acids are usually the only type which are found in naturally occurring proteins. Biosynthesis and degradation pathways of each of the 20 proteinogenic amino acids are well characterized both in prokaryotic and eukaryotic cells (see, for example, Stryer, L. Biochemistry, 3rd edition, pp. 578-590 (1988)). The “essential” amino acids (histidine, isoleucine, leucine, lysine, methionine, phenylalanine, threonine, tryptophan and valine), so-called as on account of the complexity of their biosynthesis they have to be assimilated with the food, are converted by means of simple biosynthesis pathways into the other 11 “nonessential” amino acids (alanine, arginine, asparagine, aspartate, cysteine, glutamate, glutamine, glycine, proline, serine and tyrosine). Higher animals possess the ability to synthesize some of these amino acids, but the essential amino acids have to be assimilated with the food in order that normal protein synthesis takes place.

Apart from their function in protein biosynthesis, these amino acids are interesting chemicals per se, and it has been discovered that many are used in various applications in the foodstuffs, feedstuffs, chemical, cosmetics, agriculture and pharmaceutical industries. Lysine is an important amino acid not only for the nutrition of humans, but also for monogastric animals, such as poultry and pigs. Glutamate is most often used as a flavor additive (monosodium glutamate, MSG) and to a great extent in the foodstuffs industry, as are also aspartate, phenylalanine, glycine and cysteine. Glycine, L-methionine and tryptophan are all used in the pharmaceutical industry. Glutamine, valine, leucine, isoleucine, histidine, arginine, proline, serine and alanine are used in the pharmaceutical industry and the cosmetics industry. Threonine, tryptophan and D-/L-methionine are widespread feedstuff additives (Leuchtenberger, W. (1996) Amino acids—technical production and use, pp. 466-502 in Rehm et al., (ed.) Biotechnology vol. 6, chapter 14a, VCH: Weinheim). It has been discovered that these amino acids are moreover suitable as precursors for the synthesis of synthetic amino acids and proteins, such as N-acetylcysteine, S-carboxymethyl-L-cysteine, (S)-5-hydroxytryptophan and other substances, described in Ullmann's Encyclopedia of Industrial Chemistry, vol. A2, pp. 57-97, VCH, Weinheim, 1985.

The biosynthesis of these natural amino acids in organisms which can produce them, for example bacteria, has been well characterized (for a general survey of bacterial amino acid biosynthesis and its regulation, see Umbarger, H. E. (1978) Ann. Rev. Biochem. 47: 533-606). Glutamate is synthesized by reductive animation of α-ketoglutarate, an intermediate in the citric acid cycle. Glutamine, proline and arginine are in each case produced in succession from glutamate. The biosynthesis of serine takes place in a three-step process and begins with 3-phosphoglycerate (an intermediate in glycolysis), and affords this amino acid after oxidation, transamination and hydrolysis steps. Cysteine and glycine are in each case produced from serine, namely the former by condensation of homocysteine with serine, and the latter by transfer of the side chain β-carbon atom to tetrahydrofolate, in a reaction catalyzed by serine transhydroxymethylase. Phenylalanine and tyrosine are synthesized from the precursors of the glycolysis and pentose phosphate pathway, erythrose 4-phosphate and phosphoenolpyruvate in a 9-step biosynthesis pathway, which only differs in the last two steps after the synthesis of prephenate. Tryptophan is likewise produced from these two starting molecules, but its synthesis takes place in an 11-step pathway.

Tyrosine can also be prepared from phenylalanine in a reaction catalyzed by phenylalanine hydroxylase. Alanine, valine and leucine are in each case biosynthesis products of pyruvate, the end product of the glycolysis. Aspartate is formed from oxalacetate, an intermediate of the citrate cycle. Asparagine, methionine, threonine and lysine are in each case formed by conversion of aspartate. Isoleucine is formed from threonine. In a complex 9-step pathway, the formation of histidine takes place from 5-phosphoribosyl-1-pyrophosphate, an activated sugar.

Amino acids whose amount exceeds the protein biosynthesis need of the cell cannot be stored, and instead of this are broken down, so that intermediates for the main metabolic pathways of the cell are made available (for a general survey see Stryer, L., Biochemistry, 3rd ed. chap. 21 “Amino Acid Degradation and the Urea Cycle”; pp 495-516 (1988)). Although the cell is able to convert undesired amino acids to useful metabolic intermediates, the amino acid production with respect to the energy, the precursor molecules and the enzymes necessary for their synthesis is complicated. It is therefore not surprising that amino acid biosynthesis is regulated by feedback inhibition, the presence of a certain amino acid slowing or completely ending its own production (for a general survey of the feedback mechanism in amino acid biosynthesis pathways, see Stryer, L., Biochemistry, 3rd ed., chap. 24, “Biosynthesis of Amino Acids and Heme”, pp. 575-600 (1988)). The output of a certain amino acid is therefore restricted by the amount of this amino acid in the cell.

II. Vitamins, Carotenoids, Cofactors and Nutraceuticals-Metabolism and Uses

Vitamins, carotenoids, cofactors and nutraceuticals comprise a further group of molecules. Higher animals have lost the ability to synthesize these and must thus assimilate them, although they are easily synthesized by other organisms, such as bacteria. These molecules are either biologically active molecules per se or precursors of biologically active substances, which serve as electron carriers or intermediates in a number of metabolic pathways. In addition to their nutritional value, these compounds also have a significant industrial value as colorants, antioxidants and catalysts or other processing aids. (For a general survey on the structure, activity and the industrial applications of these compounds see, for example, Ullmann's Encyclopedia of Industrial Chemistry, “Vitamins”, vol. A27, pp. 443-613, VCH: Weinheim, 1996). The term “vitamin” is known in the specialist field and comprises nutrients which are needed by an organism for normal function, but cannot be synthesized by this organism itself. The group consisting of the vitamins can comprise cofactors and nutraceutical compounds. The term “cofactor” comprises nonprotein-like compounds, which are necessary for the occurrence of normal enzyme activity. These compounds can be organic or inorganic; the cofactor molecules according to the invention are preferably organic. The term “nutraceutical” comprises food additives which are health-promoting in plants and animals, in particular humans. Examples of such molecules are vitamins, antioxidants and also certain lipids (e.g. polyunsaturated fatty acids).

Preferred fine chemicals or biosynthetic products which can be prepared in plants of the genus Tagetes, in particular in petals of the flowers of the plants of the genus Tagetes, are carotenoids, such as, for example, phytoene, lycopene, lutein, zeaxanthin, astaxanthin, canthaxanthin, echinenone, 3-hydroxyechinenone, 3′-hydroxyechinenone, adohirubin, violaxanthin and adonixanthin.

Particularly preferred carotenoids are ketocarotenoids, such as, for example, astaxanthin, canthaxanthin, echinenone, 3-hydroxyechinenone, 3′-hydroxyechinenone, adonirubin, violaxanthin and adonixanthin.

The biosynthesis of these molecules in organisms which are capable of their production, such as bacteria, has been comprehensively characterized (Ullmann's Encyclopedia of Industrial Chemistry, “Vitamins”, vol. A27, pp. 443-613, VCH: Weinheim, 1996, Michal, G. (1999) Biochemical Pathways: An Atlas of Biochemistry and Molecular Biology, John Wiley & Sons; Ong, A. S., Niki, E. and Packer, L. (1995) “Nutrition, Lipids, Health and Disease” Proceedings of the UNESCO/Confederation of Scientific and Technological Associations in Malaysia and the Society for free Radical Research—Asia, held on Sept. 1st-3rd, 1994 in Penang, Malaysia, AOCS Press, Champaign, IL X, 374 S).

Thiamine (vitamin B₁) is formed by chemical coupling of pyrimidine and thiazole units. Riboflavin (vitamin B₂) is synthesized from guanosine 5′-triphosphate (GTP) and ribose 5′-phosphate. Riboflavin is in turn employed for the synthesis of flavine mononucleotide (FMN) and flavine adenine dinucleotide (FAD). The family of compounds which together are described as “vitamin B6” (for example pyridoxine, pyridoxamine, pyridoxal 5′-phosphate and the commercially used pyridoxine hydrochloride), are all derivatives of the common structural unit 5-hydroxy-6-methylpyridine. Panthothenate (pantothenic acid, R-(+)-N-(2,4-dihydroxy-3,3-dimethyl-1-oxobutyl)-β-alanine) can be prepared either by chemical synthesis or by fermentation. The last steps in pantothenate biosynthesis consist of the ATP-driven condensation of β-alanine and pantoic acid. The enzymes responsible for the biosynthesis steps for the conversion to pantoic acid, to β-alanine and for the condensation to pantothenic acid are known. The metabolically active form of pantothenate is coenzyme A, whose biosynthesis proceeds via 5 enzymatic steps. Pantothenate, pyridoxal 5′-phosphate, cysteine and ATP are the precursors of coenzyme A. These enzymes catalyze not only the formation of pantothenate, but also the production of (R)-pantoic acid, (R)-pantolactone, (R)-panthenol (provitamin B₅), pantethein (and its derivatives) and coenzyme A.

The biosynthesis of biotin from the precursor molecule pimeloyl-CoA in microorganisms has been investigated in detail, and several of the genes involved have been identified. It has emerged that many of the corresponding proteins are involved in the Fe cluster synthesis and belong to the class of the nifS proteins. Lipoic acid is derived from octanoic acid and serves as a coenzyme in energy metabolism, where it is a constituent of the pyruvate dehydrogenase complex and of the α-ketoglutarate dehydrogenase complex. The folates are a group of substances which are all derived from folic acid, which in turn is derived from L-glutamic acid, p-amino-benzoic acid and 6-methylpterin. The biosynthesis of folic acid and its derivatives, starting from the metabolic intermediates guanosine 5′-triphosphate (GTP), L-glutamic acid and p-amino-benzoic acid, has been investigated in detail in certain microorganisms.

Corrinoids (such as the cobalamines and in particular vitamin B₁₂) and the porphyrins belong to a group of chemicals which are distinguished by a tetrapyrrole ring system. The biosynthesis of vitamin B₁₂ is sufficiently complex that it has still not been completely characterized, but in the meantime a majority of the enzymes and substrates involved is known. Nicotinic acid (nicotinate) and nicotinamide are pyridine derivatives which are also described as “niacin”. Niacin is the precursor of the important coenzyme NAD (nicotinamide adenine dinucleotide) and NADP (nicotinamide adenine dinucleotide phosphate) and their reduced forms.

The production of these compounds on the large-scale is based in the main on cell-free chemical syntheses, although some of these chemicals have also been produced by large-scale culture of microorganisms, such as riboflavin, vitamin B₆, pantothenate and biotin. Only vitamin B₁₂ is produced solely by fermentation on account of the complexity of its synthesis. In-vitro processes require a considerable expenditure of materials and time and frequently of high costs.

III. Purine, Pyrimidine, Nucleoside and Nucleotide Metabolism and Uses

Genes for purine and pyrimidine metabolism and their corresponding proteins are important targets for the therapy of tumors and virus infections. The term “purine” or “pyrimidine” comprises nitrogen-containing bases, which are a constituent of the nucleic acids, coenzymes and nucleotides. The term “nucleotide” comprises the basic structural units of the nucleic acid molecules, which comprise a nitrogen-containing base, a pentose sugar (in RNA the sugar is ribose, in DNA the sugar is D-deoxyribose) and phosphoric acid. The term “nucleoside” comprises molecules which serve as precursors of nucleotides, but which in contrast to the nucleotides contain no phosphoric acid unit. By inhibition of the biosynthesis of these molecules or their mobilization for the formation of nucleic acid molecules it is possible to inhibit the RNA and DNA synthesis; if this activity is inhibited in cancerogenic cells in a targeted manner, the division and replication ability of tumor cells can be inhibited.

There are moreover nucleotides which form no nucleic acid molecules, but serve as energy stores (i.e. AMP) or as coenzymes (i.e. FAD and NAD).

Several publications have described the use of these chemicals for these medicinal indications, in which the purine and/or pyrimidine metabolism is influenced (for example Christopherson, R. I. and Lyons, S. D. (1990) “Potent inhibitors of de novo pyrimidine and purine biosynthesis as chemotherapeutic agents”, Med. Res. Reviews 10: 505-548). Investigations on enzymes which are involved in purine and pyrimidine metabolism have concentrated on the development of new medicaments, which can be used, for example, as immunosuppressive agents or antiproliferants (Smith, J. L. “Enzymes in Nucleotide Synthesis” Curr. Opin. Struct. Biol. 5 (1995) 752-757; Biochem. Soc. Transact. 23 (1995) 877-902). The purine and pyrimidine bases, nucleosides and nucleotides, however, also have other use possibilities: as intermediates in the biosynthesis of various fine chemicals (e.g. thiamine, S-adenosylmethionine, folates or riboflavin), as energy sources for the cell (for example ATP or GTP) and, for chemicals themselves, are usually used as flavor enhancers (for example IMP or GMP) or for many medicinal applications (see, for example, Kuninaka, A., (1996) “Nucleotides and Related Compounds in Biotechnology vol. 6, Rehm et al., ed. VCH: Weinheim, pp. 561-612). Enzymes which are involved in purine, pyrimidine, nucleoside or nucleotide metabolism are also used more and more highly as targets against which chemicals for plant protection, including fungicides, herbicides and insecticides, are developed.

The metabolism of these compounds in bacteria has been characterized (for general surveys see, for example, Zalkin, H. and Dixon, J. E. (1992) “De novo purine nucleotide biosynthesis” in Progress in Nucleic Acids Research and Molecular biology, vol. 42, Academic Press, pp. 259-287; and Michal, G. (1999) “Nucleotides and Nucleosides”; chap. 8 in: Biochemical Pathways: An Atlas of Biochemistry and Molecular Biology, Wiley, New York). Purine metabolism, the subject of intensive research, is essential for the normal functioning of the cell. Defective purine metabolism in higher animals can cause serious disorders, for example gout. The purine nucleotides are synthesized from ribose 5-phosphate via a series of steps by means of the intermediate compound inosine 5′-phosphate (IMP), which leads to the production of guanosine 5′-mono-phosphate (GMP) or adenosine 5′-monophosphate (AMP), from which the triphosphate forms used as nucleotides can readily be prepared. These compounds are also used as energy stores, so that their breakdown supplies energy for many different biochemical processes in the cell. Pyrimidine biosynthesis takes place via the formation of uridine 5′-monophosphate (UMP) from ribose 5-phosphate. UMP in turn is converted to cytidine 5′-triphosphate (CTP). The deoxy forms of all nucleotides are prepared in a single-step reduction reaction of the diphosphate ribose form of the nucleotide to the diphosphate deoxyribose form of the nucleotide. After phosphorylation, these molecules can participate in DNA synthesis.

IV. Trehalose Metabolism and Uses

Trehalose consists of two glucose molecules, which are linked to one another via an α,α-1,1 bond. It is usually used in the foodstuffs industry as a sweetener, as an additive for dried or frozen foods and in drinks. It is, however, also used in the pharmaceutical industry, the cosmetics and biotechnology industry (see, for example, Nishimoto et al., (1998) U.S. Pat. No. 5,759,610; Singer, M. A. and Lindquist, S. Trends Biotech. 16 (1998) 460-467; Paiva, C. L. A. and Panek, A. D. Biotech Ann. Rev. 2 (1996) 293-314; and Shiosaka, M. J. Japan 172 (1997) 97-102). Trehalose is produced by enzymes of many microorganisms and released naturally into the surrounding medium, from which it can be recovered by processes known in the specialist field.

Particularly preferred biosynthetic products are selected from the group consisting of organic acids, proteins, nucleotides and nucleosides, both proteinogenic and nonproteinogenic amino acids, lipids and fatty acids, diols, carbohydrates, aromatic compounds, vitamins and cofactors, enzymes and proteins.

Preferred organic acids are tartaric acid, itaconic acid and diaminopimelic acid.

Preferred nucleosides and nucleotides are described, for example, in Kuninaka, A. (1996) Nucleotides and related compounds, pp. 561-612, in Biotechnology vol. 6, Rehm et al., ed. VCH: Weinheim and the citations contained therein.

Preferred biosynthetic products are furthermore lipids, saturated and unsaturated fatty acids, such as, for example, arachidonic acid, diols such as, for example, propanediol and butanediol, carbohydrates, such as, for example, hyaluronic acid and trehalose, aromatic compounds, such as, for example, aromatic amines, vanillin and indigo, vitamins and cofactors, such as are described, for example, in Ullmann's Encyclopedia of Industrial Chemistry, vol. A27, “Vitamins”, pp. 443-613 (1996) VCH: Weinheim and the citations comprised therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held on Sep. 1st-3rd, 1994 in Penang, Malysia, AOCS Press (1995)), enzyme polyketides (Cane et al. (1998) Science 282: 63-68), and all other chemicals described by Gutcho (1983) in Chemicals by Fermentation, Noyes Data Corporation, ISBN: 0818805086 and the references stated therein.

Particularly preferred genes which are expressed with the promoters according to the invention in plants of the genus Tagetes are accordingly genes selected are from the group consisting of nucleic acids encoding a protein from the biosynthesis pathway of proteinogenic and nonproteinogenic amino acids, nucleic acids encoding a protein from the biosynthesis pathway of nucleotides and nucleosides, nucleic acids encoding a protein from the biosynthesis pathway of organic acids, nucleic acids encoding a protein from the biosynthesis pathway of lipids and fatty acids, nucleic acids encoding a protein from the biosynthesis pathway of diols, nucleic acids encoding a protein from the biosynthesis pathway of carbohydrates, nucleic acids encoding a protein from the biosynthesis pathway of aromatic compound, nucleic acids encoding a protein from the biosynthesis pathway of vitamins, nucleic acids encoding a protein from the biosynthesis pathway of carotenoids, in particular ketocarotenoids, nucleic acids encoding a protein from the biosynthesis pathway of cofactors and nucleic acids encoding a protein from the biosynthesis pathway of enzymes.

Preferred fine chemicals or biosynthetic products which can be produced in plants of the genus Tagetes, in particular in petals of the flowers of the plants of the genus Tagetes, are carotenoids, such as, for example, phytoene, lycopene, lutein, zeaxanthin, astaxanthin, canthaxanthin, echinenone, 3-hydroxyechinenone, 3′-hydroxyechinenone, adonirubin, violaxanthin and adonixanthin.

Particularly preferred carotenoids are ketocarotenoids, such as, for example, astaxanthin, canthaxanthin, echinenone, 3-hydroxyechinenone, 3′-hydroxyechinenone, adonirubin, violaxanthin and adonixanthin.

Very particularly preferred genes which are expressed using the promoters according to the invention in plants of the genus Tagetes are accordingly genes which encode proteins from the biosynthesis pathway of carotenoids. Genes selected from the group consisting of nucleic acids encoding a ketolase, nucleic acids encoding a β-hydroxylase, nucleic acids encoding a β-cyclase, nucleic acids encoding an ε-cyclase, nucleic acids encoding an epoxidase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate Δ-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtISO protein, nucleic acids encoding an FtsZ protein and nucleic acids encoding a MinD protein are particularly preferred.

A ketolase is understood as meaning a protein which has the enzymatic activity to introduce a keto group on the optionally substituted β-ionone ring of carotenoids.

In particular, a ketolase is understood as meaning a protein which has the enzymatic activity to convert β-carotene to canthaxanthin.

Examples of nucleic acids encoding a ketolase, and the corresponding ketolases, are, for example, sequences from

Haematoccus pluvialis, in particular from Haematoccus pluvialis Flotow em. Wille (Accession NO: X86782; nucleic acid: SEQ ID NO: 15, protein SEQ ID NO: 16),

Haematoccus pluvialis, NIES-144 (Accession NO: D45881; nucleic acid: SEQ ID NO: 17, protein SEQ ID NO: 18),

Agrobacterium aurantiacum (Accession NO: D58420; nucleic acid: SEQ ID NO: 19, protein. SEQ ID NO: 20),

Alicaligenes spec. (Accession NO: D58422; nucleic acid: SEQ ID NO: 21, protein SEQ ID NO: 22),

Paracoccus marcusii (Accession NO: Y15112; nucleic acid: SEQ ID NO: 23, protein SEQ ID NO: 24).

Synechocystis sp. strain PC6803 (Accession NO: NP442491; nucleic acid: SEQ ID NO: 25, protein SEQ ID NO: 26).

Bradyrhizobium sp. (Accession NO: AF218415; nucleic acid: SEQ ID NO: 27, protein SEQ ID NO: 28).

Nostoc sp. strain PCC7120 (Accession NO: AP003592, BAB74888; nucleic acid: SEQ ID NO: 29, protein SEQ ID NO: 30).

Haematococcus pluvialis

(Accession NO: AF534876, AAN03484; nucleic acid: SEQ ID NO: 31, protein: SEQ ID NO: 32)

Paracoccus sp. MBIC1143

(Accession NO: D58420, P54972; nucleic acid: SEQ ID NO: 33, protein: SEQ ID NO: 34)

Brevundimonas aurantiaca

(Accession NO: AY166610, AAN86030; nucleic acid: SEQ ID NO: 35, protein: SEQ ID NO: 36)

Nodularia spumigena NSOR10

(Accession NO: AY210783, AA064399; nucleic acid: SEQ ID NO: 37, protein: SEQ ID NO: 38)

Nostoc punctiforme ATCC 29133

(Accession NO: NZ_AABC01000195, ZP_{—}00111258; nucleic acid: SEQ ID NO: 39, protein: SEQ ID NO: 40)

Nostoc punctiforme ATCC 29133

(Accession NO: NZ_AABC01000196; nucleic acid: SEQ ID NO: 41, protein: SEQ ID NO: 42)

Deinococcus radiodurans R1

(Accession NO: E75561, AE001872; nucleic acid: SEQ ID NO: 43, protein: SEQ ID NO: 44),

Synechococcus sp. WH 8102,

nucleic acid: Acc. No. NZ_AABD01000001, base pair 1,354,725-1,355,528 (SEQ ID NO: 75), protein: Acc. No. ZP_{—}00115639 (SEQ ID NO: 76) (annotated as a putative protein).

A β-cyclase is understood as meaning a protein which has the enzymatic activity to convert a terminal, linear residue of lycopene to a β-ionone ring.

In particular, a β-cyclase is understood as meaning a protein which has the enzymatic activity to convert γ-carotene to β-carotene.

Examples of β-cyclase genes are nucleic acids encoding a β-cyclase from tomato (Accession X86452) (nucleic acid: SEQ ID NO: 45, protein: SEQ ID NO: 46), and β-cyclases of the following accession numbers:

| | |
|---|---|
| S66350 | lycopene beta-cyclase (EC 5.5.1.—) - tomato |
| CAA60119 | lycopene synthase [Capsicum annuum] |
| S66349 | lycopene beta-cyclase (EC 5.5.1.—) - common tobacco |
| CAA57386 | lycopene cyclase [Nicotiana tabacum] |
| AAM21152 | lycopene beta-cyclase [Citrus sinensis] |
| AAD38049 | lycopene cyclase [Citrus × paradisi] |
| AAN86060 | lycopene cyclase [Citrus unshiu] |
| AAF44700 | lycopene beta-cyclase [Citrus sinensis] |
| AAK07430 | lycopene beta-cyclase [Adonis palaestina] |
| AAG10429 | beta cyclase [Tagetes erecta] |
| AAA81880 | lycopene cyclase |
| AAB53337 | Lycopene beta cyclase |
| AAL92175 | beta-lycopene cyclase [Sandersonia aurantiaca] |
| CAA67331 | lycopene cyclase [Narcissus pseudonarcissus] |
| AAM45381 | beta cyclase [Tagetes erecta] |
| AAO18661 | lycopene beta-cyclase [Zea mays] |
| AAG21133 | chromoplast-specific lycopene beta-cyclase [Lycopersicon esculentum] |
| AAF18989 | lycopene beta-cyclase [Daucus carota] |
| ZP_001140 | hypothetical protein [Prochlorococcus marinus str. MIT9313] |
| ZP_001050 | hypothetical protein [Prochlorococcus marinus subsp. pastoris str. |
| | CCMP1378] |
| ZP_001046 | hypothetical protein [Prochlorococcus marinus subsp. pastoris str. |
| | CCMP1378] |
| ZP_001134 | hypothetical protein [Prochlorococcus marinus str. MIT9313] |
| ZP_001150 | hypothetical protein [Synechococcus sp. WH 8102] |
| AAF10377 | lycopene cyclase [Deinococcus radiodurans] |
| BAA29250 | 393aa long hypothetical protein [Pyrococcus horikoshii] |
| BAC77673 | lycopene beta-monocyclase [marine bacterium P99-3] |
| AAL01999 | lycopene cyclase [Xanthobacter sp. Py2] |
| ZP_000190 | hypothetical protein [Chloroflexus aurantiacus] |
| ZP_000941 | hypothetical protein [Novosphingobium aromaticivorans] |
| AAF78200 | lycopene cyclase [Bradyrhizobium sp. ORS278] |
| BAB79602 | crtY [Pantoea agglomerans pv. milletiae] |
| CAA64855 | lycopene cyclase [Streptomyces griseus] |
| AAA21262 | dycopene cyclase [Pantoea agglomerans] |
| C37802 | crtY protein - Erwinia uredovora |
| BAB79602 | crtY [Pantoea agglomerans pv. milletiae] |
| AAA64980 | lycopene cyclase [Pantoea agglomerans] |
| AAC44851 | lycopene cyclase |
| BAA09593 | Lycopene cyclase [Paracoccus sp. MBIC1143] |
| ZP_000941 | hypothetical protein [Novosphingobium aromaticivorans] |
| CAB56061 | lycopene beta-cyclase [Paracoccus marcusii] |
| BAA20275 | lycopene cyclase [Erythrobacter longus] |
| ZP_000570 | hypothetical protein [Thermobifida fusca] |
| ZP_000190 | hypothetical protein [Chloroflexus aurantiacus] |
| AAK07430 | lycopene beta-cyclase [Adonis palaestina] |
| CAA67331 | lycopene cyclase [Narcissus pseudonarcissus] |
| AAB53337 | Lycopene beta cyclase |
| BAC77673 | lycopene beta-monocyclase [marine bacterium P99-3] |

A particularly preferred β-cyclase is furthermore the chromoplast-specific β-cyclase from tomato (AAG21133) (nucleic acid: SEQ. ID. No. 49; protein: SEQ. ID. No. 50).

A hydroxylase is understood as meaning a protein which has the enzymatic activity to introduce a hydroxyl group on the optionally substituted, β-ionone ring of carotenoids.

In particular, a hydroxylase is understood as meaning a protein which has the enzymatic activity to convert β-carotene to zeaxanthin or canthaxanthin to astaxanthin.

Examples of a hydroxylase gene are:

a nucleic acid encoding a hydroxylase from Haematococcus pluvialis, Accession AX038729, WO 0061764); (nucleic acid: SEQ ID NO: 49, protein: SEQ ID NO: 50),

and hydroxylases of the following accession numbers:

|emb|CAB55626.1, CAA70427.1, CAA70888.1, CAB55625.1, AF499108_{—}1, AF315289_{—}1, AF296158_{—}1, MC49443.1, NP_{—}194300.1, NP_{—}200070.1, AAG10430.1, CAC06712.1, AAM88619.1, CAC95130.1, AAL80006.1, AF162276_{—}1, AAO53295.1, AAN85601.1, CRTZ_ERWHE, CRTZ_PANAN, BAB79605.1, CRTZ_ALCSP, CRTZ_AGRAU, CAB56060.1, ZP_{—}00094836.1, AAC44852.1, BAC77670.1, NP_{—}745389.1, NP_{—}344225.1, NP_{—}849490.1, ZP_{—}00087019.1, NP_{—}503072.1, NP_{—}852012.1, NP_{—}115929.1, ZP_{—}00013255.1

A particularly preferred hydroxylase is furthermore the hydroxylase from tomato (Accession Y14810, CrtR-b2) (nucleic acid: SEQ ID NO: 51; protein: SEQ ID NO. 52).

An HMG-CoA reductase is understood as meaning a protein which has the enzymatic activity to convert 3-hydroxy-3-methyl-glutarylcoenzyme A to mevalonate.

An (E)-4-hydroxy-3-methylbut-2-enyl-diphosphate reductase is understood as meaning a protein which has the enzymatic activity to convert (E)-4-hydroxy-3-methylbut-2-enyl diphosphate to isopentenyl diphosphate and dimethylallyl diphosphates.

A 1-deoxy-D-xylose-5-phosphate synthase is understood as meaning a protein which has the enzymatic activity to convert hydroxyethyl-ThPP and glyceraldehyde 3-phosphate to 1-deoxy-D-xylose 5-phosphate.

A 1-deoxy-D-xylose-5 phosphate reductoisomerase is understood as meaning a protein which has the enzymatic activity to convert 1-deoxy-D-xylose 5-phosphate to 2-C-methyl-D-erythritol 4-phosphate.

An isopentenyl diphosphate Δ-isomerase is understood as meaning a protein which has the enzymatic activity to convert isopentenyl diphosphate to dimethylallyl phosphate.

A geranyl diphosphate synthase is understood as meaning a protein which has the enzymatic activity to convert isopentenyl diphosphate and dimethylallyl phosphate to geranyl diphosphate.

A farnesyl diphosphate synthase is understood as meaning a protein which has the enzymatic activity to convert sequentially 2 molecules of isopentenyl diphosphate using dimethylallyl diphosphate and the resulting geranyl diphosphate to farnesyl diphosphate.

A geranylgeranyl diphosphate synthase is understood as meaning a protein which has the enzymatic activity to convert farnesyl diphosphate and isopentenyl diphosphate to geranylgeranyl diphosphate.

A phytoene synthase is understood as meaning a protein which has the enzymatic activity to convert geranylgeranyl diphosphate to phytoene.

A phytoene desaturase is understood as meaning a protein which has the enzymatic activity to convert phytoene to phytofluene and/or phytofluene to ζ-carotene (zetacarotene).

A zeta-carotene desaturase is understood as meaning a protein which has the enzymatic activity to convert ζ-carotene to neurosporin and/or neurosporin to lycopene.

A crtISO protein is understood as meaning a protein which has the enzymatic activity to convert 7,9,7′,9′-tetra-cis-lycopene to all-trans-lycopene.

An FtsZ protein is understood as meaning a protein which has a cell division and plastidic division-promoting action and has homologies to tubulin proteins.

A MinD protein is understood as meaning a protein which has a multifunctional role in cell division. It is a membrane-associated ATPase and can show within the cell an oscillating motion from pole to pole.

Examples of HMG-CoA reductase genes are:

a nucleic acid encoding an HMG-CoA reductase from Arabidopsis thaliana, Accession NM_{—}106299; (nucleic acid: SEQ ID NO: 53, protein: SEQ ID NO: 54),

and further HMG-CoA reductase genes from other organisms having the following accession numbers:

P54961, P54870, P54868, P54869, O02734, P22791, P54873, P54871, P23228, P13704, P54872, Q01581, P17425, P54874, P54839, P14891, P34135, O64966, P29057, P48019, P48020, P12683, P43256, Q9XEL8, P34136, O64967, P29058, P48022, Q41437, P12684, Q00583, Q9XHL5, Q41438, Q9YAS4, O76819, O28538, Q9Y7D2, P54960, O51628, P48021, Q03163, P00347, P14773, Q12577, Q59468, P04035, O24594, P09610, Q58116, O26662, Q01237, Q01559, Q12649, O74164, O59469, P51639, Q10283, O08424, P20715, P13703, P13702, Q96UG4, Q8SQZ9, O15888, Q9TUM4, P93514, Q39628, P93081, P93080, Q944T9, Q40148, Q84MM0, Q84LS3, Q9Z9N4, Q9KLM0

Examples of (E)-4-hydroxy-3-methylbut-2-enyl diphosphate reductase genes are:

A nucleic acid encoding an (E)-4-hydroxy-3-methylbut-2-enyl diphosphate reductase from Arabidopsis thaliana (lytB/ISPH), ACCESSION AY168881, (nucleic acid: SEQ ID NO: 55, protein: SEQ ID NO:56),

and further (E)-4-hydroxy-3-methylbut-2-enyl-diphosphate reductase genes from other organisms having the following accession numbers:

T04781, AF270978_{—}1, NP_{—}485028.1, NP_{—}442089.1, NP_{—}681832:1, ZP_{—}00110421.1, ZP_{—}00071594.1, ZP_{—}00114706.1, ISPH_SYNY3, ZP_{—}00114087.1, ZP_{—}00104269.1, AF398145_{—}1, AF398146_{—}1, AAD55762.1, AF514843_{—}1, NP_{—}622970.1, NP_{—}348471.1, NP_{—}562001.1, NP_{—}223698.1, NP_{—}781941.1, ZP_{—}00080042.1, NP_{—}859669.1, NP_{—}214191.1, ZP_{—}00086191.1, ISPH_VIBCH, NP_{—}230334.1, NP_{—}742768.1, NP_{—}302306.1, ISPH_MYCLE, NP_{—}602581.1, ZP_{—}00026966.1, NP_{—}520563.1, NP_{—}253247.1, NP_{—}282047.1, ZP_{—}00038210.1, ZP_{—}00064913.1, CAA61555.1, ZP_{—}00125365.1, ISPH_ACICA, EAA24703.1, ZP_{—}00013067.1, ZP_{—}00029164.1, NP_{—}790656.1, NP_{—}217899.1, NP_{—}641592.1, NP 636532.1, NP_{—}719076.1, NP_{—}660497.1, NP_{—}422155.1, NP_{—}715446.1, ZP_{—}00090692.1, NP_{—}759496.1, ISPH_BURPS, ZP_{—}00129657.1, NP_{—}215626.1, NP_{—}335584.1, ZP_{—}00135016.1, NP_{—}789585.1, NP_{—}787770.1, NP_{—}769647.1, ZP_{—}00043336.1, NP_{—}242248.1, ZP_{—}00008555.1, NP_{—}246603.1, ZP_{—}00030951.1, NP_{—}670994.1, NP_{—}404120.1, NP_{—}540376.1, NP_{—}733653.1, NP_{—}697503.1, NP_{—}840730.1, NP_{—}274828.1, NP_{—}796916.1, ZP_{—}00123390.1, NP_{—}824386.1, NP_{—}737689.1, ZP_{—}00021222.1, NP_{—}757521.1, NP_{—}390395.1, ZP_{—}00133322.1, CAD76178.1, NP_{—}600249.1, NP_{—}454660.1, NP_{—}712601.1, NP_{—}385018.1, NP_{—}751989.1

Examples of 1-deoxy-D-xylose-5-phosphate synthase genes are:

A nucleic acid encoding a 1-deoxy-D-xylose-5-phosphate synthase from Lycopersicon esculentum, ACCESSION #AF143812 (nucleic acid: SEQ ID NO: 57, protein: SEQ ID NO: 58),

and further 1-deoxy-D-xylose-5-phosphate synthase genes from other organisms having the following accession numbers:

AF143812_{—}1, DXS_CAPAN, CAD22530.1, AF182286_{—}1, NP_{—}193291.1, T52289, AAC49368.1, AAP14353.1, D71420, DXS_ORYSA, AF443590_{—}1, BAB02345.1, CAA09804.2, NP_{—}850620.1, CAD22155.2, AAM65798.1, NP_{—}566686.1, CAD22531.1, AAC33513.1, CAC08458.1, AAG10432.1, T08140, AAP14354.1, AF428463_{—}1, ZP_{—}00010537.1, NP_{—}769291.1, AAK59424.1, NP_{—}107784.1, NP_{—}697464.1, NP_{—}540415.1, NP_{—}196699.1, NP_{—}384986.1, ZP_{—}00096461.1, ZP_{—}00013656.1, NP_{—}353769.1, BAA83576.1, ZP_{—}00005919.1, ZP_{—}00006273.1, NP_{—}420871.1, AAM48660.1, DXS_RHOCA, ZP_{—}00045608.1, ZP_{—}00031686.1, NP_{—}841218.1, ZP_{—}00022174.1, ZP_{—}00086851.1, NP_{—}742690.1, NP_{—}520342.1, ZP_{—}00082120.1, NP_{—}790545.1, ZP_{—}00125266.1, CAC17468.1, NP 252733.1, ZP_{—}00092466.1, NP_{—}439591.1, NP_{—}414954.1, NP_{—}752465.1, NP 622918.1, NP_{—}286162.1, NP_{—}836085.1, NP_{—}706308.1, ZP_{—}00081148.1, NP_{—}797065.1, NP_{—}213598.1, NP_{—}245469.1, ZP_{—}00075029.1, NP_{—}455016.1, NP_{—}230536.1, NP_{—}459417.1, NP_{—}274863.1, NP_{—}283402.1, NP_{—}759318.1, NP_{—}406652.1, DXS_SYNLE, DXS_SYNP7, NP_{—}440409.1, ZP_{—}00067331.1, ZP_{—}00122853.1, NP_{—}717142.1, ZP_{—}00104889.1, NP_{—}243645.1, NP_{—}681412.1, DXS_SYNEL, NP_{—}637787.1, DXS_CHLTE, ZP_{—}00129863.1, NP_{—}661241.1, DXS_XANCP, NP_{—}470738.1, NP_{—}484643.1, ZP_{—}00108360.1, NP_{—}833890.1, NP_{—}846629.1, NP_{—}658213.1, NP_{—}642879.1, ZP_{—}00039479.1, ZP_{—}00060584.1, ZP_{—}00041364.1, ZP_{—}00117779.1, NP_{—}299528.1

Examples of 1-deoxy-D-xylose-5-phosphate reductoisomerase genes are:

A nucleic acid encoding a 1-deoxy-D-xylose-5-phosphate reductoisomerase from Arabidopsis thaliana, ACCESSION #AF148852, (nucleic acid: SEQ ID NO: 59, protein: SEQ ID NO: 60),

and further 1-deoxy-D-xylose-5-phosphate reductoisomerase genes from other organisms having the following accession numbers:

AF148852, AY084775, AY054682, AY050802, AY045634, AY081453, AY091405, AY098952, AJ242588, AB009053, AY202991, NP_{—}201085.1, T52570, AF331705_{—}1, BAB16915.1, AF367205_{—}1, AF250235_{—}1, CAC03581.1, CAD22156.1, AF182287_{—}1, DXR_MENP₁, ZP_{—}00071219.1, NP_{—}488391.1, ZP_{—}00111307.1, DXR_SYNLE, AAP56260.1, NP_{—}681831.1, NP_{—}442113.1, ZP_{—}00115071.1, ZP_{—}00105106.1, ZP_{—}00113484.1, NP_{—}833540.1, NP_{—}657789.1, NP_{—}661031.1, DXR_BACHD, NP_{—}833080.1, NP_{—}845693.1, NP_{—}562610.1, NP_{—}623020.1, NP_{—}810915.1, NP_{—}243287.1, ZP_{—}00118743.1, NP_{—}464842.1, NP_{—}470690.1, ZP_{—}00082201.1, NP_{—}781898.1, ZP_{—}00123667.1, NP_{—}348420.1, NP_{—}604221.1, ZP_{—}00053349.1, ZP_{—}00064941.1, NP_{—}246927.1, NP_{—}389537.1, ZP_{—}00102576.1, NP_{—}519531.1, AF124757_{—}19, DXR_ZYMMO, NP_{—}713472.1, NP_{—}459225.1, NP_{—}454827.1, ZP_{—}00045738.1, NP_{—}743754.1, DXR_PSEPK, ZP_{—}00130352.1, NP_{—}702530.1, NP_{—}841744.1, NP_{—}438967.1, AF514841_{—}1, NP_{—}706118.1, ZP_{—}00125845.1, NP_{—}404661.1, NP_{—}285867.1, NP_{—}240064.1, NP_{—}414715.1, ZP_{—}00094058.1, NP_{—}791365.1, ZP_{—}00012448.1, ZP_{—}00015132.1, ZP_{—}00091545.1, NP_{—}629822.1, NP_{—}771495.1, NP_{—}798691.1, NP_{—}231885.1, NP_{—}252340.1, ZP_{—}00022353.1, NP_{—}355549.1, NP_{—}420724.1, ZP_{—}00085169.1, EAA17616.1, NP_{—}273242.1, NP_{—}219574.1, NP_{—}387094.1, NP_{—}296721.1, ZP_{—}00004209.1, NP_{—}823739.1, NP_{—}282934.1, BAA77848.1, NP_{—}660577.1, NP_{—}760741.1, NP_{—}641750.1, NP_{—}636741.1, NP_{—}829309.1, NP_{—}298338.1, NP_{—}444964.1, NP_{—}717246.1, NP_{—}224545.1, ZP_{—}00038451.1, DXR_KITGR, NP_{—}778563.1.

Examples of isopentenyl diphosphate Δ-isomerase genes are:

A nucleic acid encoding an isopentenyl diphosphate Δ-isomerase from Adonis palaestina clone ApIPI28, (ipiAa1), ACCESSION #AF188060, published by Cunningham, F. X. Jr. and Gantt, E.: Identification of multi-gene families encoding isopentenyl diphosphate isomerase in plants by heterologous complementation in Escherichia coli, Plant Cell Physiol. 41 (1), 119-123 (2000) (nucleic acid: SEQ ID NO: 61, protein: SEQ ID NO: 62),

and further isopentenyl diphosphate Δ-isomerase genes from other organisms having the following accession numbers:

Q38929, O48964, Q39472, Q13907, O35586, P58044, O42641, O35760, Q10132, P15496, Q9YB30, Q8YNH4, Q42553, O27997, P50740, O51627, O48965, Q8KFR5, Q39471, Q39664, Q9RVE2, Q01335, Q9HHE4, Q9BXS1, Q9 KWF6, Q9CIF5, Q88WB6, Q92BX2, Q8Y7A5, Q8TT35 Q9KK75, Q8NN99, Q8XD58, Q8FE75, Q46822, Q9HP40, P72002, P26173, Q9Z5D3, Q8Z3X9, Q8ZM82, Q9X7Q6, O13504, Q9HFW8, Q8NJL9, Q9UUQ1, Q9NH02, Q9M6K9, Q9M6K5, Q9FXR6, O81691, Q9S7C4, Q8S3L8, Q9M592, Q9M6K3, Q9M6K7, Q9FV48, Q9LLB6, Q9AVJ1, Q9AVG8, Q9M6K6, Q9AVJ5, Q9M6K2, Q9AYS5, Q9M6K8, Q9AVG7, Q8S3L7, Q8W250, Q941E1, Q9AVI8, Q9AYS6, Q9SAY0, Q9M6K4, Q8GVZ0, Q84RZ8, Q8KZ12, Q8KZ66, Q8FND7, Q88QC9, Q8BFZ6, BAC26382, CAD94476.

Examples of geranyl diphosphate synthase genes are:

A nucleic acid encoding a geranyl diphosphate synthase from Arabidopsis thaliana, ACCESSION #Y17376, Bouvier, F., Suire, C., d'Harlingue, A., Backhaus, R. A. and Camara, B.; Molecular cloning of geranyl diphosphate synthase and compartmentation of monoterpene synthesis in plant cells, Plant J. 24 (2), 241-252 (2000) (nucleic acid: SEQ ID NO: 63, protein: SEQ ID NO: 64),

and further geranyl diphosphate synthase genes from other organisms having the following accession numbers:

Q9FT89, Q8LKJ2, Q9FSW8, Q8LKJ3, Q9SBR3, Q9SBR4, Q9FET8, Q8LKJ1, Q84LG1, Q9JK86

Examples of farnesyl diphosphate synthase genes are:

A nucleic acid encoding a farnesyl diphosphate synthase from Arabidopsis thaliana (FPS1), ACCESSION #U80605, published by Cunillera, N., Arro, M., Delourme, D., Karst, F., Boronat, A. and Ferrer, A.: Arabidopsis thaliana contains two differentially expressed farnesyl-diphosphate synthase genes, J. Biol. Chem. 271 (13), 7774-7780 (1996), (nucleic acid: SEQ ID NO: 65, protein: SEQ ID NO:66),

and further farnesyl diphosphate synthase genes from other organisms having the following accession numbers:

P53799, P37268, Q02769, Q09152, P49351, O24241, Q43315, P49352, O24242, P49350, P08836, P14324, P49349, P08524, O66952, Q08291, P54383, Q45220, P57537, Q8K9A0, P22939, P45204, O66126, P55539, Q9SWH9, Q9AVI7, Q9FRX2, Q9AYS7, Q941E8, Q9FXR9, Q9ZWF6, Q9FXR8, Q9AR37, O50009, Q941E9, Q8RVK7, Q8RVQ7, O04882, Q93RA8, Q93RB0, Q93RB4, Q93RB5, Q93RB3, Q93RB1, Q93RB2, Q920E5.

Examples of geranylgeranyl diphosphate synthase genes are:

A nucleic acid encoding a geranylgeranyl diphosphate synthase from Sinaps alba, ACCESSION #X98795, published by Bonk, M., Hoffmann, B., Von Lintig, J., Schledz, M., Al-Babili, S., Hobeika, E., Kleinig, H. and Beyer, P.: Chloroplast import of four carotenoid biosynthetic enzymes in vitro reveals differential fates prior to membrane binding and oligomeric assembly, Eur. J. Biochem. 247 (3), 942-950 (1997), (nucleic acid: SEQ ID NO: 67, protein: SEQ ID NO: 68),

and further geranylgeranyl diphosphate synthase genes from other organisms having the following accession numbers:

P22873, P34802, P56966, P80042, Q42698, Q92236, O95749, Q9WTN0, Q50727, P24322, P39464, Q9FXR3, Q9AYN2, Q9FXR2, Q9AVG6, Q9FRW4, Q9SXZ5, Q9AVJ7, Q9AYN1, Q9AVJ4, Q9FXR7, Q8LSC5, Q9AVJ6, Q8LSC4, Q9AVJ3, Q9SSU0, Q9SXZ6, Q9SST9Q9AVJ0, Q9AVI9, Q9FRW3, Q9FXR5, Q941F0, Q9FRX1, Q9K567, Q93RA9, Q93QX8, CAD95619, EAA31459

Examples of phytoene synthase genes are:

A nucleic acid encoding a phytoene synthase from Erwinia uredovora, ACCESSION # D90087; published by Misawa, N., Nakagawa, M., Kobayashi, K., Yamano, S., Izawa, Y., Nakamura, K. and Harashima, K.: Elucidation of the Erwinia uredovora carotenoid biosynthetic pathway by functional analysis of gene products expressed in Escherichia coli; J. Bacteriol. 172 (12), 6704-6712 (1990), (nucleic acid: SEQ ID NO: 69, protein: SEQ ID NO: 70),

and further phytoene synthase genes from other organisms having the following accession numbers:

CAB39693, BAC69364, AAF10440, CAA45350, BAA20384, AAM72615, BAC09112, CAA48922, P_{—}001091, CAB84588, AAF41518, CAA48155, AAD38051, AAF33237, AAG10427, AAA34187, BAB73532, CAC19567, AAM62787, CAA55391, AAB65697, AAM45379, CAC27383, AAA32836, AAK07735, BAA84763, P_{—}000205, AAB60314, P_{—}001163, P_{—}000718, MB71428, AAA34153, AAK07734, CAA42969, CAD76176, CAA68575, P_{—}000130, P_{—}001142, CAA47625, CAA85775, BAC14416, CAA79957, BAC76563, P_{—}000242, P_{—}000551, AAL02001, AAK15621, CAB94795, AAA91951, P_{—}000448

Examples of phytoene desaturase genes are:

A nucleic acid encoding a phytoene desaturase from Erwinia uredovora, ACCESSION # D90087; published by Misawa, N., Nakagawa, M., Kobayashi, K., Yamano, S., Izawa, Y., Nakamura, K. and Harashima, K.: Elucidation of the Erwinia uredovora carotenoid biosynthetic pathway by functional analysis of gene products expressed in Escherichia coli; J. Bacteriol. 172 (12), 6704-6712 (1990), (nucleic acid: SEQ ID NO: 71, protein: SEQ ID NO: 72),

and further phytoene desaturase genes from other organisms having the following accession numbers:

AAL15300, A39597, CAA42573, AAK51545, BAB08179, CAA48195, BAB82461, AAK92625, CAA55392, AAG10426, AAD02489, M024235, AAC12846, AAA99519, AAL38046, CAA60479, CAA75094, ZP_{—}001041, ZP_{—}001163, CAA39004, CAA44452, ZP_{—}001142, ZP_{—}000718, BAB82462, AAM45380, CAB56040, ZP_{—}001091, BAC09113, AAP79175, AAL80005, AAM72642, AAM72043, ZP_{—}000745, ZP_{—}001141, BAC07889, CAD55814, ZP_{—}001041, CAD27442, CAE00192, ZP_{—}001163, ZP_{—}000197, BAA18400, AAG10425, ZP_{—}001119, AAF13698, 2121278A, AAB35386, AAM02462, BAB68552, CAC85667, AAK51557, CAA12062, AAG51402, AAM63349, AAF85796, BAB74081, AAA91161, CAB56041, MC48983, AAG14399, CAB65434, BAB73487, ZP_{—}001117, ZP_{—}000448, CAB39695, CAD76175, BAC69363, AAM17934, ZP_{—}000171, AAF65586, ZP_{—}000748, BAC07074, ZP_{—}001133, CAA64853, BAB74484, ZP_{—}001156, AAF23289, AAG28703, AAP09348, AAM71569, BAB69140, ZP_{—}000130, AAF41516, AAG18866, CAD95940, NP_{—}656310, AAG10645, ZP_{—}000276, ZP_{—}000192, ZP_{—}000186, AAM94364, EAA31371, ZP_{—}000612, BAC75676, AAF65582

Examples of zeta-carotene desaturase genes are:

A nucleic acid encoding a zeta-carotene desaturase from Narcissus pseudonarcissus, ACCESSION #AJ224683, published by AI-Babili, S., Oelschlegel, J. and Beyer, P.: A cDNA encoding for beta carotene desaturase (Accession No. AJ224683) from Narcissus pseudonarcissus L. (PGR98-103), Plant Physiol. 117, 719-719 (1998), (nucleic acid: SEQ ID NO: 73, protein: SEQ ID NO: 74),

and further zeta-carotene desaturase genes from other organisms having the following accession numbers:

Q9R6×4, Q38893, Q9SMJ3, Q9SE20, Q9ZTP4, O49901, P74306, Q9FV46, Q9RCT2, ZDS_NARPS, BAB68552.1, CAC85667.1, AF372617_{—}1, ZDS_TARER, CAD55814.1, CAD27442.1, 2121278A, ZDS_CAPAN, ZDS_LYCES, NP_{—}187138.1, AAM63349.1, ZDS_ARATH, AAA91161.1, ZDS_MAIZE, AAG14399.1, NP_{—}441720.1, NP_{—}486422.1, ZP_{—}00111920.1, CAB56041.1, ZP_{—}00074512.1, ZP_{—}00116357.1, NP_{—}681127.1, ZP_{—}00114185.1, ZP_{—}00104126.1, CAB65434.1, NP_{—}662300.1

Examples of crtISO genes are:

A nucleic acid encoding a crtISO from Lycopersicon esculentum; ACCESSION #AF416727, published by Isaacson, T., Ronen, G., Zamir, D. and Hirschberg, J.: Cloning of tangerine from tomato reveals a carotenoid isomerase essential for the production of beta-carotene and xanthophylls in plants; Plant Cell 14 (2), 333-342 (2002), (nucleic acid: SEQ ID NO: 75, protein: SEQ ID NO: 76),

and further crtISO genes from other organisms having the following accession numbers:

AAM53952

Examples of FtsZ genes are:

A nucleic acid encoding an FtsZ from Tagetes erecta, ACCESSION #AF251346, published by Moehs, C. P., Tian, L., Osteryoung, K. W. and Dellapenna, D.: Analysis of carotenoid biosynthetic gene expression during marigold petal development Plant Mol. Biol. 45 (3), 281-293 (2001), (nucleic acid: SEQ ID NO: 77, protein: SEQ ID NO: 78),

and further FtsZ genes from other organisms having the following accession numbers:

CAB89286.1, AF205858_{—}1, NP_{—}200339.1, CAB89287.1, CAB41987.1, AAA82068.1, T06774, AF383876_{—}1, BAC57986.1, CAD22047.1, BAB91150.1, ZP_{—}00072546.1, NP_{—}440816.1, T51092, NP_{—}683172.1, BAA85116.1, NP_{—}487898.1, JC4289, BM82871.1, NP_{—}781763.1, BAC57987.1, ZP_{—}00111461.1, T51088, NP_{—}190843.1, ZP_{—}00060035.1, NP_{—}846285.1, AAL07180.1, NP_{—}243424.1, NP_{—}833626.1, AAN04561.1, AAN04557.1, CAD22048.1, T51089, NP_{—}692394.1, NP_{—}623237.1, NP_{—}565839.1, T51090, CAA07676.1, NP_{—}113397.1, T51087, CAC44257.1, E84778, ZP_{—}00105267.1, BAA82091.1, ZP_{—}00112790.1, BAA96782.1, NP_{—}348319.1, NP_{—}471472.1, ZP_{—}00115870.1, NP_{—}465556.1, NP_{—}389412.1, BM82090.1, NP_{—}562681.1, MM22891.1, NP_{—}371710.1, NP_{—}764416.1, CAB95028.1, FTSZ_STRGR, AF120117_{—}1, NP_{—}827300.1, JE0282, NP_{—}626341.1, MC45639.1, NP 785689.1, NP_{—}336679.1, NP_{—}738660.1, ZP_{—}00057764.1, AAC32265.1, NP_{—}814733.1, FTSZ_MYCKA, NP_{—}216666.1, CAA75616.1, NP_{—}301700.1, NP_{—}601357.1, ZP_{—}00046269.1, CM70158.1, ZP_{—}00037834.1, NP_{—}268026.1, FTSZ_ENTHR, NP_{—}787643.1, NP_{—}346105.1, MC32264.1, JC5548, AAC95440.1, NP_{—}710793.1, NP_{—}687509.1, NP_{—}269594.1, AAC32266.1, NP_{—}720988.1, NP_{—}657875.1, ZP_{—}00094865.1, ZP_{—}00080499.1, ZP_{—}00043589.1, JC7087, NP_{—}660559.1, AAC46069.1, AF179611_{—}14, AAC44223.1, NP_{—}404201.1.

Examples of MinD genes are:

A nucleic acid encoding a MinD from Tagetes erecta, ACCESSION #AF251019, published by Moehs, C. P., Tian, L., Osteryoung, K. W. and Dellapenna, D.: Analysis of carotenoid biosynthetic gene expression during marigold petal development; Plant Mol. Biol. 45 (3), 281-293 (2001), (nucleic acid: SEQ ID NO: 79, protein: SEQ ID NO: 80),

and further MinD genes having the following accession numbers:

NP_{—}197790.1, BAA90628.1, NP_{—}038435.1, NP_{—}045875.1, AAN33031.1, NP_{—}050910.1, CAB53105.1, NP_{—}050687.1, NP_{—}682807.1, NP_{—}487496.1, ZP_{—}00111708.1, ZP_{—}00071109.1, NP_{—}442592.1, NP_{—}603083.1, NP_{—}782631.1, ZP_{—}00097367.1, ZP_{—}00104319.1, NP_{—}294476.1, NP_{—}622555.1, NP_{—}563054.1, NP_{—}347881.1, ZP_{—}00113908.1, NP_{—}834154.1, NP_{—}658480.1, ZP_{—}00059858.1, NP_{—}470915.1, NP_{—}243893.1, NP_{—}465069.1, ZP_{—}00116155.1, NP_{—}390677.1, NP_{—}692970.1, NP_{—}298610.1, NP_{—}207129.1, ZP_{—}00038874.1, NP_{—}778791.1, NP_{—}223033.1, NP_{—}641561.1, NP_{—}636499.1, ZP_{—}00088714.1, NP_{—}213595.1, NP_{—}743889.1, NP_{—}231594.1, ZP_{—}00085067.1, NP 797252.1, ZP_{—}00136593.1, NP_{—}251934.1, NP_{—}405629.1, NP_{—}759144.1, ZP_{—}00102939.1, NP_{—}793645.1, NP_{—}699517.1, NP_{—}460771.1, NP_{—}860754.1, NP_{—}456322.1, NP_{—}718163.1, NP_{—}229666.1, NP_{—}357356.1, NP_{—}541904.1, NP_{—}287414.1, NP_{—}660660.1, ZP_{—}00128273.1, NP_{—}103411.1, NP_{—}785789.1, NP_{—}715361.1, AF149810_{—}1, NP_{—}841854.1, NP_{—}437893.1, ZP_{—}00022726.1, EM24844.1, ZP_{—}00029547.1, NP_{—}521484.1, NP_{—}240148.1, NP_{—}770852.1, AF3459082, NP_{—}777923.1, ZP_{—}00048879.1, NP_{—}579340.1, NP_{—}143455.1, NP_{—}126254.1, NP_{—}142573.1, NP_{—}613505.1, NP_{—}127112.1, NP 712786.1, NP_{—}578214.1, NP_{—}069530.1, NP_{—}247526.1, MA85593.1, NP 212403.1, NP_{—}782258.1, ZP_{—}00058694.1, NP_{—}247137.1, NP_{—}219149.1, NP_{—}276946.1, NP_{—}614522.1, ZP_{—}00019288.1, CAD78330.1

The invention further relates to a genetically modified plant of the genus Tagetes, the genetic modification leading to an increasing or causing of the expression rate of at least one gene in comparison with the wild-type and being due to the regulation of the expression of this gene in the plant by the promoters according to the invention.

As mentioned above, “expression activity” is understood according to the invention as meaning the amounts of protein formed in a certain time by the promoter, that is the expression rate.

“Specific expression activity” is understood according to the invention as meaning the amount of protein per promoter formed in a certain time by the promoter.

In a “caused expression activity” or “caused expression rate” in relation to a gene in comparison with the wild-type, in comparison with the wild-type the formation of a protein is thus caused which was not present in the wild-type of the plant of the genus Tagetes.

For example, wild-type plants of the genus Tagetes have no ketolase gene. The regulation of the expression of the ketolase gene in the plant by the promoters according to the invention thus leads to a causing of the expression rate.

In an “increased expression activity” or “increased expression rate” in relation to a gene in comparison with the wild-type, in comparison with the wild-type the amount of protein formed in a certain time is thus increased in the plant of the genus Tagetes.

For example, wild-type plants of the genus Tagetes have a hydroxylase gene. The regulation of the expression of the hydroxylase gene in the plant by the promoters according to the invention thus leads to an increasing of the expression rate. In a preferred embodiment of the genetically modified plants of the genus Tagetes according to the invention, the regulation of the expression of genes in the plant is achieved by means of the promoters according to the invention, in that

a) one or more promoters according to the invention is inserted into the genome of the plant such that the expression of one or more endogenous genes takes place under the control of the inserted promoters according to the invention or

b) one or more genes is inserted into the genome of the plant such that the expression of one or more of the inserted genes takes place under the control of the endogenous promoters according to the invention or

c) one or more nucleic acid constructs comprising at least one promoter according to the invention and, functionally linked, one or more genes to be expressed are inserted into the plant.

In a preferred embodiment, according to feature c) one or more nucleic acid constructs comprising at least one promoter according to the invention and, functionally linked, one or more genes to be expressed are inserted into the plant. The integration of the nucleic acid constructs into the plant of the genus Tagetes can in this case take place intrachromosomally or extrachromosomally.

Preferred promoters according to the invention and preferred genes to be expressed (effect genes) are described above.

By way of example, the production of the genetically modified plants of the genus Tagetes having an increased or caused expression rate of an effect gene is described below.

The transformation can be carried out individually or by means of multiple constructs in the combinations of genetic modifications.

The production of the transgenic plants is preferably carried out by transformation of the starting plants, using a nucleic acid construct which comprises at least one of the promoters according to the invention described above, which are functionally linked to an effect gene to be expressed and, if appropriate, to further regulation signals.

These nucleic acid constructs, in which the promoters according to the invention and effect genes are functionally linked, are also called expression cassettes below.

The expression cassettes can comprise further regulation signals, that is regulative nucleic acid sequences, which control the expression of the effect genes in the host cell. According to a preferred embodiment, an expression cassette comprises upstream, i.e. at the 5′-end of the coding sequence, at least one promoter according to the invention and downstream, i.e. at the 3′-end, a polyadenylation signal and, if appropriate, further regulatory elements which are operatively linked with the intermediate coding sequence of the effect gene for at least one of the genes described above.

An operative linkage is understood as meaning the sequential arrangement of promoter, coding sequence, terminator and, if appropriate, further regulative elements in such a way that each of the regulative elements can fulfill its function in the expression of the coding sequence as intended.

By way of example, the preferred nucleic acid constructs, expression cassettes and vectors for plants and processes for the production of transgenic plants, and the transgenic plants of the genus Tagetes themselves are described below.

The sequences which are preferred, but not restricted thereto, for the operative linkage are targeting sequences for the guaranteeing of the subcellular location in the apoplast, in the vacuoles, in plastids, in the mitochondrium, in the endoplasmatic reticulum (ER), in the cell nucleus, in elaioplasts or other compartments and translation enhancers such as the 5′-guiding sequence of the tobacco mosaic virus (Gallie et al., Nucl. Acids Res. 15 (1987), 8693-8711).

The production of an expression cassette is preferably carried out by fusion of at least one promoter according to the invention with at least one gene, preferably with one of the effect genes described above, and preferably a nucleic acid inserted between promoter and nucleic acid sequence, which codes for a plastid-specific transit peptide, and a polyadenylation signal according to customary recombination and cloning techniques, such as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and also in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

The preferably inserted nucleic acids, encoding a plastidic transit peptide, guarantee location in plastids and in particular in chromoplasts.

Expression cassettes can also be used whose nucleic acid sequence codes for an effect gene-product fusion protein, a part of the fusion protein being a transit peptide which controls the translocation of the polypeptide. Specific transit peptides, which are removed enzymatically from the effect gene product part after translocation of the effect genes to the chromoplasts, are preferred for the chromoplasts.

The transit peptide which is derived from the plastidic Nicotiana tabacum transketolase or another transit peptide (e.g. the transit peptide of the small subunit of the Rubisco (rbcS) or of the ferredoxin NADP oxidoreductase and the isopentenyl pyrophosphate isomerase-2 or its functional equivalent) is particularly preferred.

Nucleic acid sequences of three cassettes of the plastid transit peptide of the plastidic transketolase from tobacco in three reading frames as KpnI/BamHI fragments with an ATG codon in the NcoI cleavage site are particularly preferred:

pTP09
KpnI_GGTACCATGGCGTCTTCTTCTTCTCTCACTCTCTCTCAAGCTATCCTCTCTC
GTTCTGTCCCTCGCCATGGCTCTGCCTCTTCTTCTCAACTTTCCCCTTCTTCTCTC
ACTTTTTCCGGCCTTAAATCCAATCCCAATATCACCACCTCCCGCCGCCGTACTCC
TTCCTCCGCCGCCGCCGCCGCCGTCGTAAGGTCACCGGCGATTCGTGCCTCAGC
TGCAACCGAAACCATAGAGAAAACTGAGACTGCGGGATCC_BamHI

pTP10
KpnI_GGTACCATGGCGTCTTCTTCTTCTCTCACTCTCTCTCAAGCTATCCTCTCTC
GTTCTGTCCCTCGCCATGGCTCTGCCTCTTCTTCTCAACTTTCCCCTTCTTCTCTC
ACTTTTTCCGGCCTTAAATCCAATCCCAATATCACCACCTCCCGCCGCCGTACTCC
TTCCTCCGCCGCCGCCGCCGCCGTCGTAAGGTCACCGGCGATTCGTGCCTCAGC
TGCAACCGAAACCATAGAGAAAACTGAGACTGCGCTGGATCC_BamHI

pTP11
KpnI_GGTACCATGGCGTCTTCTTCTTCTCTCACTCTCTCTCAAGCTATCCTCTCTC
GTTCTGTCCCTCGCCATGGCTCTGCCTCTTCTTCTCAACTTTCCCCTTCTTCTCTC
ACTTTTTCCGGCCTTAAATCCAATCCCAATATCACCACCTCCCGCCGCCGTACTCC
TTCCTCCGCCGCCGCCGCCGCCGTCGTAAGGTCACCGGCGATTCGTGCCTCAGC
TGCAACCGAAACCATAGAGAAAACTGAGACTGCGGGGATCC_BamHI

Further examples of a plastidic transit peptide are the transit peptide of the plastidic isopentenyl pyrophosphate isomerase-2 (IPP-2) from Arabisopsis thaliana and the transit peptide of the small subunit of the ribulose bisphosphate carboxylase (rbcS) from pea (Guerineau, F, Woolston, S, Brooks, L, Mullineaux, P (1988) An expression cassette for targeting foreign proteins into the chloroplasts. Nucl. Acids Res. 16: 11380).

The nucleic acids according to the invention can be prepared synthetically or obtained naturally or comprise a mixture of synthetic and natural nucleic acid constituents, and consist of various heterologous gene sections of various organisms.

As described above, synthetic nucleotide sequences with codons which are preferred from plants are preferred. These codons preferred from plants can be determined from codons having the highest protein frequency, which are expressed in the most interesting plant species.

In the preparation of an expression cassette, various DNA fragments can be manipulated in order to obtain a nucleotide sequence which expediently reads in the correct direction and and which is equipped with a correct reading frame. For the connection of the DNA fragments to one another, adapters or linkers can be added to the fragments.

Expediently, the promoter and the terminator regions can be provided in the transcription direction with a linker or polylinker which comprises one or more restriction sites for the insertion of this sequence. As a rule, the linker has 1 to 10, usually 1 to 8, preferably 2 to 6, restriction sites. In general, the linker within the regulatory areas has a size of less than 100 bp, frequently less than 60 bp, but at least 5 bp. The promoter can be either native or homologous, or foreign or heterologous for the host plant. The expression cassette preferably comprises, in the 5′-3′ transcription direction, the promoter, a coding nucleic acid sequence or a nucleic acid construct and a region for transcriptional termination. Various termination regions are mutually arbitrarily exchangeable.

Examples of a terminator are the ³⁵S terminator (Guerineau et al. (1988) Nucl Acids Res. 16: 11380), the nos terminator (Depicker A, Stachel S, Dhaese P, Zambryski P, Goodman H M. Nopaline synthase: transcript mapping and DNA sequence. J Mol Appl Genet. 1982; 1(6):561-73) or the ocs terminator (Gielen, J, de Beuckeleer, M, Seurinck, J, Debroek, H, de Greve, H, Lemmers, M, van Montagu, M, Schell, J (1984) The complete sequence of the TL-DNA of the Agrobacterium tumefaciens plasmid pTiAch5. EMBO J. 3: 835-846).

Furthermore, manipulations which make available suitable restriction cleavage sites or remove the superfluous DNA or restriction cleavage sites can be employed. Where insertions, deletions or substitutions such as, for example, transitions and transversions are possible, in vitro mutagenesis, “primer repair”, restriction or ligation can be used.

With suitable manipulations, such as, for example, restriction, “chewing-back” or filling of overhangs for “blunt ends”, complementary ends of the fragments can be made available for ligation.

Preferred polyadenylation signals are plant polyadenylation signals, preferably those which essentially correspond to T-DNA polyadenylation signals from Agrobacterium tumefaciens, in particular of gene 3 of the T-DNA (octopine synthase) of the Ti plasmid pTiACH5 (Gielen et al., EMBO J. 3 (1984), 835 ff) or functional equivalents.

The transposition of foreign genes into the genome of a plant is described as transformation.

To this end, methods known per se for the transformation and regeneration of plants from plant tissues or plant cells can be utilized for transient or stable transformation.

Suitable methods for the transformation of plants are protoplast transformation by polyethylene glycol-induced DNA uptake, the biolistic process using the gene gun—the “particle bombardment” method, electroporation, the incubation of dry embryos in DNA-containing solution, microinjection and gene transfer mediated by Agrobacterium, described above. Said processes are described, for example, in B. Jenes et al., Techniques for Gene Transfer, in: Transgenic Plants, Vol. 1, Engineering and Utilization, edited by S. D. Kung and R. Wu, Academic Press (1993), 128-143, and in Potrykus, Annu. Rev. Plant Physiol. Plant Molec. Biol. 42 (1991), 205-225.

Preferably, the construct to be expressed is cloned in a vector which is suitable for transforming Agrobacterium tumefaciens, for example pBin19 (Bevan et al., Nucl. Acids Res. 12 (1984), 8711) or particularly preferably pSUN2, pSUN3, pSUN4 or pSUN5 (WO 02/00900).

Agrobacteria transformed using an expression plasmid can be used in a known manner for the transformation of plants, e.g. by bathing wounded leaves or leaf pieces in an Agrobacteria solution and subsequently culturing in suitable media.

For the preferred production of genetically modified plants, also called transgenic plants below, the fused expression cassette is cloned in a vector, for example pBin19 or in particular pSUN5 and pSUN3, which is suitable to be transformed into Agrobacterium tumefaciens. Agrobacteria transformed using such a vector can then be used in a known manner for the transformation of plants, in particular of crop plants, by, for example, bathing wounded leaves or pieces of leaf in an Agrobacteria solution and subsequently culturing in suitable media.

The transformation of plants by Agrobacteria is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38. From the transformed cells of the wounded leaves or pieces of leaf, transgenic plants can be regenerated in a known manner which comprise one or more genes integrated into the expression cassette.

For the transformation of a host plant using one or more effect genes according to the invention, an expression cassette is incorporated into a recombinant vector as an insertion whose vector DNA comprises additional functional regulation signals, for example sequences for replication or integration. Suitable vectors are described, inter alia, in “Methods in Plant Molecular Biology and Biotechnology” (CRC Press), Chap. 6/7, pp. 71-119 (1993).

Using the recombination and cloning techniques cited above, the expression cassettes can be cloned into suitable vectors which make possible their proliferation, for example in E. coli. Suitable cloning vectors are, inter alia, pJIT117 (Guerineau et al. (1988) Nucl. Acids Res. 16:11380), pBR332, pUC series, M13mp series and pACYC184. Particularly suitable are binary vectors, which can replicate both in E. coli and in Agrobacteria.

The invention therefore furthermore relates to a genetically modified plant of the genus Tagetes, comprising a promoter according to the invention and, functionally linked, to a gene to be expressed, with the proviso that genes from plants of the genus Tagetes, which are expressed in wild-type plants of the genus Tagetes by the respective promoter, are excluded.

Preferred promoters and preferred effect genes according to the invention are described above.

Particularly preferably, effect genes are selected from the group consisting of nucleic acids encoding a ketolase, nucleic acids encoding a β-hydroxylase, nucleic acids encoding a β-cyclase, nucleic acids encoding an ε-cyclase, nucleic acids encoding an epoxidase, nucleic acids encoding an HMG-CoA reductase, nucleic acids encoding an (E)-4-hydroxy-3-methylbut-2-enyl diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate Δ-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtISO protein, nucleic acids encoding an FtsZ protein and nucleic acids encoding a MinD protein.

Preferred, genetically modified plants of the genus Tagetes are Marigold, Tagetes erecta, Tagetes patula, Tagetes lucida, Tagetes pringlei, Tagetes palmeri, Tagetes minuta or Tagetes campanulata.

By means of the promoters according to the invention, it is possible with the aid of the processes according to the invention described above to regulate, in the genetically modified plants of the genus Tagetes according to the invention described above, the metabolic pathways to specific biosynthetic products.

To this end, for example, metabolic pathways which lead to a specific biosynthetic product are strengthened by causing or increasing the transcription rate or expression rate of genes of this biosynthesis pathway by the increased amount of protein leading to an increased total activity of these proteins of the desired biosynthesis pathway and thus by an increased metabolic flow to the desired biosynthetic product.

Depending on the desired biosynthetic product, the transcription rate or expression rate of various genes must be increased or reduced. As a rule, it is advantageous to modify the transcription rate or expression rate of several genes, i.e. to increase the transcription rate or expression rate of a combination of genes and/or to reduce the transcription rate or expression rate of a combination of genes.

In the genetically modified plants according to the invention, at least one increased or caused expression rate of a gene is to be attributed to a promoter according to the invention.

Further, additionally modified, i.e. additionally increased or additionally reduced, expression rate of further genes in genetically modified plants can, but do not have to, be attributed to the promoters according to the invention.

The invention therefore relates to a process for the preparation of biosynthetic products by culturing genetically modified plants of the genus Tagetes according to the invention.

The invention relates in particular to a process for the production of carotenoids by culturing genetically modified plants of the genus Tagetes according to the invention, wherein the genes to be expressed are selected from the group consisting of nucleic acids encoding a ketolase, nucleic acids encoding a β-hydroxylase, nucleic acids encoding a β-cyclase, nucleic acids encoding an ε-cyclase, nucleic acids encoding an epoxidase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate Δ-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtlSO protein, nucleic acids encoding an FtsZ protein and nucleic acids encoding a MinD protein.

The carotenoids are preferably selected from the group consisting of phytoene, phytofluene, lycopene, lutein, zeaxanthin, astaxanthin, canthaxanthin, echinenone, 3-hydroxyechinenone, 3′-hydroxyechinenone, adonirubin, violaxanthin and adonixanthin.

In particular, the invention furthermore relates to a process for the production of ketocarotenoids by culturing genetically modified plants of the genus Tagetes according to the invention, wherein the genes to be expressed are selected from the group consisting of nucleic acids encoding a ketolase, nucleic acids encoding a β-hydroxylase, nucleic acids encoding a β-cyclase, nucleic acids encoding an α-cyclase, nucleic acids encoding an epoxidase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl diphosphate Δ-isomerase, nucleic acids encoding a geranyl diphosphate synthase, nucleic acids encoding a farnesyl diphosphate synthase, nucleic acids encoding a geranylgeranyl diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtlSO protein, nucleic acids encoding an FtsZ protein and nucleic acids encoding a MinD protein.

The ketocarotenoids are preferably selected from the group consisting of astaxanthin, canthaxanthin, echinenone, 3-hydroxyechinenone, 3′-hydroxyechinenone, adonirubin, violaxanthin and adonixanthin.

In the process according to the invention for the preparation of biosynthetic products, in particular carotenoids, preferably ketocarotenoids, a harvesting of the plants and an isolation of the biosynthetic products, in particular carotenoids, preferably ketocarotenoids, from the plants, preferably from the petals of the plants, is preferably added to the culturing step of the genetically modified plants.

The genetically modified plants of the genus Tagetes are grown in a manner known per se on nutrient media and appropriately harvested.

The isolation of ketocarotenoids from the harvested flower leaves is carried out, for example in a manner known per se, for example by drying and subsequent extraction and, if appropriate, further chemical or physical purification processes, such as, for example, precipitation methods, crystallography, thermal separation processes, such as rectifying processes or physical separation processes, such as, for example, chromatography. The isolation of ketocarotenoids from the flower leaves is carried out, for example, preferably by means of organic solvents such as acetone, hexane, heptane, ether or tert-methyl butyl ether.

Further isolation processes of ketocarotenoids, in particular from flower leaves, are described, for example, in Egger and Kleinig (Phytochemistry (1967) 6, 437-440) and Egger (Phytochemistry (1965) 4, 609-618).

A particularly preferred ketocarotenoid is astaxanthin.

The ketocarotenoids are obtained in the process according to the invention in flower leaves in the form of their mono- or diesters with fatty acids. Some proven fatty acids are, for example, myristic acid, palmitic acid, stearic acid, oleic acid, linolenic acid, and lauric acid (Kamata and Simpson (1987) Comp. Biochem. Physiol. Vol. 86B(3), 587-591).

Genetically modified plants or plant parts according to the invention consumable by humans and animals, such as, in particular, flower leaves having an increased content of biosynthetic products, in particular carotenoids, in particular ketocarotenoids, in particular astaxanthin, can also be used, for example, directly or after processing known per se, as foodstuffs or feedstuffs or as feed and food supplements.

Furthermore, the genetically modified plants can be used for the production of biosynthetic product-, in particular carotenoid-, in particular ketocarotenoid-, in particular astaxanthin-containing extracts and/or for the production of feed and food supplements, and of cosmetics and pharmaceuticals.

The genetically modified plants of the genus Tagetes have, in comparison with the wild-type, an increased content of the desired biosynthetic products, in particular carotenoids, in particular ketocarotenoids, in particular astaxanthin.

An increased content is in this case also understood as meaning a caused content of ketocarotenoids, or astaxanthin.

The invention is illustrated by the examples which now follow, but is not restricted to these:

General experimental conditions:

Sequence analysis of recombinant DNA

The sequencing of recombinant DNA molecules was carried out using a laser fluorescence DNA sequencer from Licor (marketing by MWG Biotech, Ebersbach) according to the method of Sanger (Sanger et al., Proc. Natl. Acad. Sci. USA 74 (1977), 5463-5467).

### EXAMPLE 1

Amplification of a DNA Which Encodes the Entire Primary Sequence of the NOST Ketolase from Nostoc sp. PCC 7120

The DNA which codes for the NOST ketolase from Nostoc sp. PCC 7120 was amplified by means of PCR from Nostoc sp. PCC 7120 (strain of the “Pasteur Culture Collection of Cyanobacterium”).

For the preparation of genomic DNA from a suspension culture of Nostoc sp. PCC 7120 which had been grown for 1 week with continuous illumination and constant shaking (150 rpm) at 25° C. in BG 11 medium (1.5 g/l of NaNO3, 0.04 g/l of K2PO4×3H₂O, 0.075 g/l of MgSO4×H₂O, 0.036 g/l of CaCl2×2H₂O, 0.006 g/l of citric acid, 0.006 g/l of ferric ammonium citrate, 0.001 g/l of EDTA disodium magnesium, 0.04 g/l of Na2CO3, 1 ml of Trace Metal Mix “A5+Co” (2.86 g/l H₃BO3, 1.81 g/l of MnCl2×4H2o; 0.222 g/l of ZnSO4×7H2o, 0.39 g/l of NaMoO4×2H₂O, 0.079 g/l of CuSO4×5H₂O, 0.0494 g/l of Co(NO3)2×6H₂O)), the cells were harvested by centrifugation, frozen in liquid nitrogen and pulverized in a mortar.

Protocol for DNA isolation from Nostoc PCC7120:

The bacterial cells from a 10 ml liquid culture were pelleted by centrifugation at 8000 rpm for 10 minutes. Subsequently, the bacterial cells were pulverized and ground in liquid nitrogen using a mortar. The cell material was resuspended in 1 ml 10 mM Tris HCl (pH 7.5) and transferred to an Eppendorf reaction vessel (2 ml volume). After addition of 100 μl of proteinase K (concentration: 20 mg/ml), the cell suspension was incubated for 3 hours at 37° C. Subsequently, the suspension was extracted using 500 μl of phenol. After centrifugation at 13 000 upm for 5 minutes, the upper, aqueous phase was transferred to a new 2 ml Eppendorf reaction vessel. The extraction with phenol was repeated 3 times. The DNA was precipitated by addition of a 1/10 volume of 3 M sodium acetate (pH 5.2) and 0.6 volume of isopropanol and subsequently washed with 70% ethanol. The DNA pellet was dried at room temperature, taken up in 25 μl of water and dissolved at 65° C. with heating.

The nucleic acid encoding a ketolase from Nostoc PCC 7120 was amplified by means of “polymerase chain reaction” (PCR) from Nostoc sp. PCC 7120 using a sense-specific primer (NOSTF, SEQ ID No. 79) and an antisense-specific primer (NOSTG SEQ ID No. 80).

The PCR conditions used were as below:

The PCR for the amplification of the DNA which codes for a ketolase protein consisting of the entire primary sequence was carried out in a 50 ul reaction batch, in which were comprised:

1 ul of a Nostoc sp. PCC 7120 DNA (prepared as described above)

0.25 mM dNTPs

0.2 mM NOSTF (SEQ ID No. 79)

0.2 mM NOSTG (SEQ ID No. 80)

5 ul of 10×PCR buffer (TAKARA)

0.25 ul of R Taq polymerase (TAKARA)

25.8 ul of of dist. water

The PCR was carried out under the following cycle conditions:

1×94° C. 2 minutes

35×94° C. 1 minute

55° C. 1 minutes

72° C. 3 minutes

1×72° C. 10 minutes

The PCR amplification with SEQ ID No. 79 and SEQ ID No. 80 resulted in an 805 bp fragment, which codes for a protein consisting of the entire primary sequence (SEQ ID No. 81). Using standard methods, the amplificate was cloned in the PCR cloning vector pGEM-T (Promega) and the clone pNOSTF-G was obtained.

Sequencing of the clone pNOSTF-G using the M13F and the M13R primer confirmed a sequence which is identical with the DNA sequence from 88,886-89,662 of the database entry AP003592. This nucleotide sequence was reproduced in an independent amplification experiment and thus represents the nucleotide sequence in the Nostoc sp. PCC 7120 used.

### EXAMPLE 2

Amplification of a DNA Which Encodes the Total Primary Sequence of the NP196 Ketolase from Nostoc punctiforme ATCC 29133

The DNA which codes for the NP196 ketolase from Nostoc punctiforme ATCC 29133 was amplified by means of PCR from Nostoc punctiforme ATCC 29133 (strain of the “American Type Culture Collection”).

For the preparation of genomic DNA from a suspension culture of Nostoc punctiforme ATCC 29133 which had been grown for 1 week with continuous illumination and constant shaking (150 rpm) at 25° C. in BG 11 medium (1.5 g/l of NaNO₃, 0.04 g/l of K₂PO₄×3H₂O, 0.075 g/l of MgSO₄×H₂O, 0.036 g/l of CaCl₂×2H₂O, 0.006 g/l of citric acid, 0.006 g/l of ferric ammonium citrate, 0.001 g/l of EDTA disodium magnesium, 0.04 g/l of Na₂CO₃, 1 ml of Trace Metal Mix “A5+Co” (2.86 g/l of H₃BO₃, 1.81 g/l of MnCl₂×4H₂O, 0.222 g/l of ZnSO₄×7H₂O, 0.39 g/l of NaMoO₄×2H₂O, 0.079 g/l of CuSO₄×5H₂O, 0.0494 g/l of Co(NO₃)₂×6H₂O)), the cells were harvested by centrifugation, frozen in liquid nitrogen and pulverized in a mortar.

Protocol for the isolation of DNA from Nostoc punctiforme ATCC 29133:

The bacterial cells from a 10 ml liquid culture were pelleted by centrifugation at 8000 rpm for 10 minutes. Subsequently, the bacterial cells were pulverized and ground in liquid nitrogen with a mortar. The cell material was resuspended in 1 ml 10 mM Tris HCl (pH 7.5) and transferred to an Eppendorf reaction vessel (2 ml volume). After addition of 100 μl of proteinase K (concentration: 20 mg/ml), the cell suspension was incubated for 3 hours at 37° C. subsequently, the suspension was extracted with 500 μl of phenol. After centrifugation at 13 000 upm for 5 minutes, the upper, aqueous phase was transferred to a new 2 ml Eppendorf reaction vessel. The extraction with phenol was repeated 3 times. The DNA was precipitated by addition of a 1/10 volume of 3 M sodium acetate (pH 5.2) and 0.6 volume of isopropanol and subsequently washed with 70% ethanol. The DNA pellet was dried at room temperature, taken up in 25 μl of water and dissolved with heating at 65° C.

The nucleic acid encoding a ketolase from Nostoc punctiforme ATCC 29133 was amplified by means of “polymerase chain reaction” (PCR) of Nostoc punctiforme ATCC 29133 using a sense-specific primer (NP196-1, SEQ ID No. 82) and an antisense-specific primer (NP196-2 SEQ ID No. 83).

The PCR conditions were as follows:

The PCR for the amplification of the DNA which codes for a ketolase protein consisting of the total primary sequence was carried out in a 50 ul reaction batch, in which were comprised:

1 ul of a Nostoc punctiforme ATCC 29133 DNA (prepared as described above)

0.25 mM dNTPs

0.2 mM NP196-1 (SEQ ID No. 82)

0.2 mM NP196-2 (SEQ ID No. 83)

5 ul of 10×PCR buffer (TAKARA)

0.25 ul of R Taq polymerase (TAKARA)

25.8 ul of dist. water

The PCR was carried out under the following cycle conditions:

1×94° C. 2 minutes

35×94° C. 1 minute

55° C. 1 minutes

72° C. 3 minutes

1×72° C. 10 minutes

The PCR amplification with SEQ ID No. 82 and SEQ ID No. 83 resulted in a 792 bp fragment which coded for a protein consisting of the entire primary sequence (NP196, SEQ ID No. 84). Using standard methods, the amplificate was cloned in the PCR cloning vector pCR 2.1 (Invitrogen) and the clone pNP196 was obtained.

Sequencing of the clone pNP196 using the M13F and the M13R primer confirmed a sequence which is identical to the DNA sequence from 140,571-139,810 of the database entry NZ_AABC01000196 (inversely oriented to the published database entry) with the exception that G in position 140,571 was replaced by A in order to produce a standard start codon ATG. This nucleotide sequence was reproduced in an independent amplification experiment and thus represents the nucleotide sequence in the Nostoc punctiforme ATCC 29133 used.

This clone pNP196 was therefore used for cloning in the expression vector pJIT117 (Guerineau et al. 1988, Nucl. Acids Res. 16: 11380).

pJIT117 was modified by replacing the 35S terminator by the OCS terminator (octopine synthase) of the Ti plasmid pTi15955 of Agrobacterium tumefaciens (database entry X00493 from position 12,541-12,350, Gielen et al. (1984) EMBO J. 3 835-846).

The DNA fragment which comprises the OCS terminator region was prepared by means of PCR using the plasmid pHELLSGATE (database entry AJ311874, Wesley et al. (2001) Plant J. 27 581-590, isolated from E. coli according to standard methods) and the primer OCS-1 (SEQ ID No. 85) and OCS-2 (SEQ ID No. 86).

The PCR conditions were as follows:

The PCR for the amplification of the DNA which comprises the octopine synthase (OCS) terminator region (SEQ ID No. 87) was carried out in a 50 ul reaction batch, in which were comprised:

100 ng of pHELLSGATE plasmid DNA

0.25 mM dNTPs

0.2 mM OCS-1 (SEQ ID No. 85)

0.2 mM OCS-2 (SEQ ID No. 86)

5 ul of 10×PCR buffer (Stratagene)

0.25 ul of Pfu polymerase (Stratagene)

28.8 ul of dist. water

The PCR was carried out under the following cycle conditions:

1×94° C. 2 minutes

35×94° C. 1 minute

50° C. 1 minute

72° C. 1 minute

1×72° C. 10 minutes

The 210 bp amplificate was cloned using standard methods in the PCR cloning vector pCR 2.1 (Invitrogen) and the plasmid pOCS was obtained.

Sequencing of the clone pOCS confirmed a sequence which corresponded to a sequence section on the Ti plasmid pTi15955 of Agrobacterium tumefaciens (database entry X00493) from position 12,541 to 12,350.

The cloning was carried out by isolation of the 210 bp SalI-XhoI fragment from pOCS and ligation in the SalI-XhoI-cleaved vector pJIT117.

This clone is called pJO and was therefore used for cloning in the expression vector pJONP196.

The cloning was carried out by isolation of the 782 Bp SphI fragment from pNP196 and ligation in the SphI-cleaved vector pJO. The clone which comprises the NP196 ketolase of Nostoc punctiforme in the correct orientation as the N-terminal translational fusion with the rbcS transit peptide is called pJONP196.

### EXAMPLE 3

Preparation of Expression Vectors for the Flower-Specific Expression of the NP196 Ketolase from Nostoc punctiforme ATCC 29133 in Tagetes erecta

The expression of the NP196 ketolase from Nostoc punctiforme in Tagetes erecta was carried out with the transit peptide rbcS from pea (Anderson et al. 1986, Biochem J. 240:709-715). The expression was carried out under the control of the flower-specific promoter EPSPS from Petunia hybrida (database entry M37029: nucleotide region 7-1787; Benfey et al. (1990) Plant Cell 2: 849-856).

The DNA fragment which comprises the EPSPS promoter region (SEQ ID No. 88) from Petunia hybrida was prepared by means of PCR using genomic DNA (isolated according to standard methods from Petunia hybrida) and the primer EPSPS-1 (SEQ ID No. 89) and EPSPS-2 (SEQ ID No. 90).

The PCR conditions were as follows:

The PCR for the amplification of the DNA which comprises the EPSPS promoter fragment (database entry M37029: nucleotide region 7-1787) was carried out in a 50 μl reaction batch, in which were comprised:

100 ng of genomic DNA from A. thaliana

0.25 mM dNTPs

0.2 mM EPSPS-1 (SEQ ID No. 89)

0.2 mM EPSPS-2 (SEQ ID No. 90)

5 ul of 10×PCR buffer (Stratagene)

0.25 ul of Pfu polymerase (Stratagene)

28.8 ul of dist. water

The PCR was carried out under the following cycle conditions:

1×94° C. 2 minutes

35×94° C. 1 minute

50° C. 1 minute

72° C. 2 minute

1×72° C. 10 minutes

The 1773 Bp amplificate was cloned using standard methods in the PCR cloning vector pCR 2.1 (Invitrogen) and the plasmid pEPSPS was obtained.

Sequencing of the clone pEPSPS confirmed a sequence which only differed by two deletions (bases ctaagtttcagga in position 46-58 of the sequence M37029; bases aaaaatat in position 1422-1429 of the sequence M37029) and the base exchanges (T instead of G in position 1447 of the sequence M37029; A instead of C in position 1525 of the sequence M37029; A instead of G in position 1627 of the sequence M37029) from the published EPSPS sequence (database entry M37029: nucleotide region 7-1787). The two deletions and the two base exchanges in the positions 1447 and 1627 of the sequence M37029 were reproduced in an independent amplification experiment and thus represent the actual nucleotide sequence in the Petunia hybrida plants used.

The clone pEPSPS was therefore used for cloning in the expression vector pJONP196.

The cloning was carried out by isolation of the 1763 bp SacI-HindIII fragment from pEPSPS and ligation in the SacI-HindIII-cleaved vector pJONP196. The clone which comprises the promoter EPSPS instead of the original promoter d35S is called pJOESP:NP196. This expression cassette comprises the fragment NP196 in the correct orientation as the N-terminal fusion with the rbcS transit peptide.

For the preparation of the expression vector MSP107, the 2,961 KB bp SacI-XhoI fragment from pJOESP:NP196 was ligated with the SacI-XhoI-cleaved vector pSUN3. MSP107 comprises fragment EPSPS the EPSPS promoter (1761 bp), fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

The preparation of an expression vector for the Agrobacterium-mediated transformation of the EPSPS-controlled NP196 ketolase from Nostoc punctiforme in Tagetes erecta was carried out using the binary vector pSUN5 (WO02/00900).

For the preparation of the expression vector MSP108, the 2,961 KB bp SacI-XhoI fragment from pJOESP:NP196 was ligated with the SacI-XhoI cleaved vector pSUN5. MSP108 comprises fragment EPSPS the EPSPS promoter (1761 bp), fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

### EXAMPLE 4

Amplification of a DNA Which Encodes the Total Primary Sequence of the NP195 Ketolase from Nostoc punctiforme ATCC 29133.

The DNA which codes for the NP195 ketolase from Nostoc punctiforme ATCC 29133 was amplified by means of PCR from Nostoc punctiforme ATCC 29133 (strain of the “American Type Culture Collection”). The preparation of genomic DNA from a suspension culture of Nostoc punctiforme ATCC 29133 was described in Example 19.

The nucleic acid encoding a ketolase from Nostoc punctiforme ATCC 29133 was amplified by means of “polymerase chain reaction” (PCR) from Nostoc punctiforme ATCC 29133 using a sense-specific primer (NP195-1, SEQ ID No. 91) and an antisense-specific primer (NP195-2 SEQ ID No. 92).

The PCR conditions were as follows:

The PCR for the amplification of the DNA which codes for a ketolase protein consisting of the total primary sequence was carried out in a 50 ul reaction batch, in which was comprised:

1 ul of a Nostoc punctiforme ATCC 29133 DNA (prepared as described above)

0.25 mM dNTPs

0.2 mM NP195-1 (SEQ ID No. 91)

0.2 mM NP195-2 (SEQ ID No. 92)

5 ul of 10×PCR buffer (TAKARA)

0.25 ul of R Taq polymerase (TAKARA)

25.8 ul of dist. water

The PCR was carried out under the following cycle conditions:

1×94° C. 2 minutes

35×94° C. 1 minute

55° C. 1 minutes

72° C. 3 minutes

1×72° C. 10 minutes

The PCR amplification with SEQ ID No. 91 and SEQ ID No. 92 resulted in an 819 bp fragment which codes for a protein consisting of the total primary sequence (NP195, SEQ ID No. 93). Using standard methods, the amplificate was cloned in the PCR cloning vector pCR 2.1 (Invitrogen) and the clone pNP195 obtained.

Sequencing of the clone pNP195 with the M13F and the M13R primer confirmed a sequence which is identical with the DNA sequence from 55,604-56,392 of the database entry NZ_AABC010001965, with the exception that T in position 55,604 was replaced by A in order to produce a standard start codon ATG. This nucleotide sequence was reproduced in an independent amplification experiment and thus represents the nucleotide sequence in the Nostoc punctiforme ATCC 29133 used.

This clone pNP195 was therefore used for cloning in the expression vector pJ0 (described in Example 6). The cloning was carried out by isolation of the 809 bp SphI fragment from pNP195 and ligation in the SphI-cleaved vector pJO. The clone which comprises the NP195 ketolase from Nostoc punctiforme in the correct orientation as the N-terminal translational fusion with the rbcS transit peptide is called pJONP195.

### EXAMPLE 5

Amplification of a DNA Which Encodes the Entire Primary Sequence of the NODK Ketolase from Nodularia spumignea NSOR10.

The DNA which codes for the ketolase from Nodularia spumignea NSOR10 was amplified by means of PCR from Nodularia spumignea NSOR10.

For the preparation of genomic DNA from a suspension culture of Nodularia spumignea NSOR10, which was grown for 1 week with continuous light and constant shaking (150 rpm) at 25° C. in BG 11 medium (1.5 g/l of NaNO₃, 0.04 g/l of K₂PO₄×3H₂O, 0.075 g/l of MgSO₄×H₂O, 0.036 g/l of CaCl₂×2H₂O, 0.006 g/l of citric acid, 0.006 g/l of ferric ammonium citrate, 0.001 g/l of EDTA disodium magnesium, 0.04 g/l of Na₂CO₃, 1 ml of trace metal mix “A5+Co” (2.86 g/l of H₃BO₃, 1.81 g/l of MnCl₂×4H₂O, 0.222 g/l of ZnSO₄×7H₂O, 0.39 g/l of NaMoO₄×2H₂O, 0.079 g/l of CuSO₄×5H₂O, 0.0494 g/l of Co(NO₃)₂×6H₂O), the cells were harvested by centrifugation, frozen in liquid nitrogen and pulverized in a mortar.

Protocol for DNA isolation from Nodularia spumignea NSOR10:

The bacterial cells were pelleted from a 10 ml liquid culture by centrifugation at 8000 rpm for 10 minutes. The bacterial cells were subsequently pulverized and ground in liquid nitrogen using a mortar. The cell material was resuspended in 1 ml of 10 mM Tris-HCl (pH 7.5) and transferred to an Eppendorf reaction vessel (2 ml volume). After addition of 100 μl of proteinase K (concentration: 20 mg/ml), the cell suspension was incubated for 3 hours at 37° C. Subsequently, the suspension was extracted with 500 μl of phenol. After centrifugation at 13 000 rpm for 5 minutes, the upper, aqueous phase was transferred to a new 2 ml Eppendorf reaction vessel. The extraction with phenol was repeated 3 times. The DNA was precipitated by addition of a 1/10 volume of 3 M sodium acetate (pH 5.2) and 0.6 volume of isopropanol and subsequently washed with 70% ethanol. The DNA pellet was dried at room temperature, taken up in 25 μl of water and dissolved with heating to 65° C.

The nucleic acid encoding a ketolase from Nodularia spumignea NSOR10 was amplified by means of “polymerase chain reaction” (PCR) from Nodularia spumignea NSOR10 using a sense-specific primer (NODK-1, SEQ ID No. 94) and an antisense-specific primer (NODK-2 SEQ ID No. 95).

Die PCR conditions were as follows:

The PCR for the amplification of the DNA which encodes a ketolase protein consisting of the entire primary sequence was carried out in a 50 ul reaction batch, in which was comprised:

1 ul of a Nodularia spumignea NSOR10 DNA (prepared as described above)

0.25 mM dNTPs

0.2 mM NODK-1 (SEQ ID No. 94)

0.2 mM NODK-2 (SEQ ID No. 95)

5 ul of 10×PCR buffer (TAKARA)

0.25 ul of R Taq polymerase (TAKARA)

25.8 ul of dist. water

The PCR was carried out under the following cycle conditions:

| | | |
|---|---|---|
| 1X | 94° C. | 2 minutes |
| 35X | 94° C. | 1 minute |
| | 55° C. | 1 minutes |
| | 72° C. | 3 minutes |
| 1X | 72° C. | 10 minutes |

The PCR amplification with SEQ ID No. 94 and SEQ ID No. 95 resulted in a 720 bp fragment which codes for a protein consisting of the entire primary sequence (NODK, SEQ ID No. 96). Using standard methods, the amplificate was cloned in the PCR cloning vector pCR 2.1 (Invitrogen) and the clone pNODK was obtained.

Sequencing of the clone pNODK with the M13F and the M13R primers confirmed a sequence which is identical to the DNA sequence from 2130-2819 of the database entry AY210783 (orientated inversely to the published database entry). This nucleotide sequence was reproduced in an independent amplification experiment and thus represents the nucleotide sequence in the Nodularia spumignea NSOR10 used.

This clone pNODK was therefore used for cloning in the expression vector pJO (described in Example 6). Cloning was carried out by isolation of the 710 bp SphI fragment from pNODK and ligation in the SphI-cleaved vector pJO. The clone which comprises the NODK ketolase of Nodularia spumignea in the correct orientation as the N-terminal translational fusion with the rbcS transit peptide is called pJONODK.

### EXAMPLE 6

Production of Expression Vectors for the Flower-Specific Expression of the NODK-Ketolase from Nodularia spumignea NSOR10 in Lycopersicon esculentum and Tagetes erecta.

The expression of the NODK ketolase from Nodularia spumignea NSOR10 in L. esculentum and Tagetes erecta was carried out using the transit peptide rbcS from pea (Anderson et al. 1986, Biochem J. 240:709-715). Expression was carried out under the control of the flower-specific promoter EPSPS from Petunia hybrida (database entry M37029: nucleotide region 7-1787; Benfey et al. (1990) Plant Cell 2: 849-856).

The clone pEPSPS (described in Example 8) was therefore used for cloning in the expression vector pJONODK (described in Example 12).

Cloning was carried out by isolation of the 1763 bp SacI-HindIII fragment from pEPSPS and ligation in the SacI-HindIII-cleaved vector pJONODK. The clone which comprises the promoter EPSPS instead of the original promoter d35S is called pJOESP:NODK. This expression cassette comprises the fragment NODK in the correct orientation as the N-terminal fusion with the rbcS transit peptide.

The production of an expression vector for the Agrobacterium-mediated transformation of the EPSPS-controlled NODK ketolase from Nodularia spumignea NSOR10 in L. esculentum was carried out using the binary vector pSUN3 (WO02/00900).

For the production of the expression vector MSP115, the 2,889 KB bp SacI-XhoI fragment from pJOESP:NODK was ligated with the SacI-XhoI-cleaved vector pSUN3. MSP 115 comprises fragment EPSPS the EPSPS promoter (1761 bp), fragment rbcS TP FRAGMENT the rbcS transit peptide from pea (194 bp), fragment NODK KETO CDS (690 bp), coding for the Nodularia spumignea NSOR10 NODK ketolase, fragment OCS terminator (192 bp) the polyadenylation signal of octopine synthase.

The production of an expression vector for the Agrobacterium-mediated transformation of the EPSPS-controlled NODK ketolase from Nodularia spumignea NSOR10 in Tagetes erecta was carried out using the binary vector pSUN5 (WO02/00900).

For the production of the expression vector MSP116, the 2,889 KB bp SacI-XhoI fragment from pJOESP:NODK was ligated with the SacI-XhoI-cleaved vector pSUN5. MSP 116 comprises fragment EPSPS the EPSPS promoter (1761 bp), fragment rbcS TP FRAGMENT the rbcS transit peptide from pea (194 bp), fragment NODK KETO CDS (690 bp), coding for the Nodularia spumignea NSOR10 NODK ketolase, fragment OCS terminator (192 bp) the polyadenylation signal of octopine synthase.

### EXAMPLE 6A

Preparation of Expression Vectors for the Flower-Specific Expression of the NP196 Ketolase from Nostoc punctiforme ATCC 29133 in Lycopersicon esculentum and Tagetes erecta.

The expression of the NP196 ketolase from Nostoc punctiforme in Tagetes erecta was carried out with the transit peptide rbcS from pea (Anderson et al. 1986, Biochem J. 240:709-715). The expression was carried out under the control of the flower-specific promoter PDS (phytoene desaturase) from Lycopersicon esculentum (database entry U46919).

The DNA fragment which comprises the PDS promoter region (SEQ ID No. 100) from Lycopersicon esculentum was prepared by means of PCR using genomic DNA (isolated from Lycopersicon esculentum according to standard methods) and the primer PDS-1 (SEQ ID No. 98) and PDS-2 (SEQ ID No. 99).

The PCR conditions were as follows:

The PCR for the amplification of the DNA which comprises the PDS promoter fragment was carried out in a 50 μl reaction batch, in which was comprised:

100 ng of genomic DNA from Lycopersicon esculentum

0.25 mM dNTPs

0.2 mM PDS-1 (SEQ ID No. 98)

0.2 mM PDS-2 (SEQ ID No. 99)

5 ul of 10× PCR buffer (Stratagene)

0.25 ul of Pfu polymerase (Stratagene)

28.8 ul of dist. water

The PCR was carried out under the following cycle conditions:

| | | |
|---|---|---|
| 1X | 94° C. | 2 minutes |
| 35X | 94° C. | 1 minute |
| | 50° C. | 1 minute |
| | 72° C. | 2 minute |
| 1X | 72° C. | 10 minutes |

The 2096 bp amplificate was cloned using standard methods in the PCR cloning vector pCR 2.1 (Invitrogen) and the plasmid pPDS obtained.

The clone pPDS was therefore used for cloning in the expression vector pJOEPS:NP196 (described in Example 3).

The cloning was carried out by isolation of the 2094 bp Ecl136II-SmaI fragment from pPDS and ligation in the Ecl136II-HindIII-cleaved vector pJOEPS:NP196. The HindIII cleavage site of the vector was converted beforehand to a “blunt-end” cleavage site by treatment with the Klenow enzyme. The clone which comprises the promoter PDS instead of the original promoter EPSPS is called pJOPDS:NP196. This expression cassette comprises the fragment NP196 in the correct orientation as the N-terminal fusion with the rbcS transit peptide.

The preparation of an expression vector for the Agrobacterium-mediated transformation of the PDS-controlled NP196 ketolase from Nostoc punctiforme in L. esculentum was carried out using the binary vector pSUN3 (WO02/00900).

For the preparation of the expression vector MSP117, the 3.3 KB bp Ecl136II-XhoI fragment from pJOPDS:NP196 was ligated with the Ecl136II-XhoI-cleaved vector pSUN3. MSP117 comprises fragment PDS the PDS promoter, fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

The preparation of an expression vector for the Agrobacterium-mediated transformation of the PDS-controlled NP196 ketolase from Nostoc punctiforme in Tagetes erecta was carried out using the binary vector pSUN5 (WO02/00900). For the preparation of the expression vector MSP118, the 3.3 KB bp Ecl136II-XhoI fragment from pJOPDS:NP196 was ligated with the Ecl136II-XhoI-cleaved vector pSUN5. MSP118 comprises fragment PDS the PDS promoter, fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

### EXAMPLE 6B

Preparation of Expression Vectors for the Flower-Specific Expression of the NP196 Ketolase from Nostoc punctiforme ATCC 29133 in Lycopersicon esculentum and Tagetes erecta

The expression of the NP196 ketolase from Nostoc punctiforme in Tagetes erecta was carried out with the transit peptide rbcS from pea (Anderson et al. 1986, Biochem J. 240:709-715). The expression was carried out under the control of the flower-specific promoter B-GENE (chromoplast-specific lycopene B-cyclase) from Lycopersicon esculentum (database entry AAZ51517).

The DNA fragment which comprises the B-GENE promoter region (SEQ ID No. 103) from Lycopersicon esculentum was prepared by means of PCR using genomic DNA (isolated from Lycopersicon esculentum according to standard methods) and the primer BGEN-1 (SEQ ID No. 101) and BGEN-2 (SEQ ID No. 102).

The PCR conditions were as follows:

The PCR for the amplification of the DNA which comprises the B-GENE promoter fragment was carried out in a 50 μl reaction batch, in which was comprised:

100 ng of genomic DNA from Lycopersicon esculentum

0.25 mM dNTPs

0.2 mM BGEN-1 (SEQ ID No. 101)

0.2 mM BGEN-2 (SEQ ID No. 102)

5 ul of 10× PCR buffer (Stratagene)

0.25 ul of Pfu polymerase (Stratagene)

28.8 ul of dist. water

The PCR was carried out under the following cycle conditions:

| | | |
|---|---|---|
| 1X | 94° C. | 2 minutes |
| 35X | 94° C. | 1 minute |
| | 50° C. | 1 minute |
| | 72° C. | 2 minute |
| 1X | 72° C. | 10 minutes |

The 1222 bp amplificate was cloned using standard methods in the PCR cloning vector pCR 2.1 (Invitrogen) and the plasmid pB-GENE obtained.

The clone pB-GENE was therefore used for cloning in the expression vector pJOEPS:NP196 (described in Example 3).

The cloning was carried out by isolation of the 1222 bp SacI-HindIII fragment from pB-GENE and ligation in the SacI-HindIII-cleaved vector pJOEPS:NP196. The clone which comprises the promoter B-GENE instead of the original promoter EPSPS is called pJOBGEN:NP196. This expression cassette comprises the fragment NP196 in the correct orientation as the N-terminal fusion with the rbcS transit peptide.

The preparation of an expression vector for the Agrobacterium-mediated transformation of the PDS-controlled NP196 ketolase from Nostoc punctiforme in L. esculentum was carried out using the binary vector pSUN3 (WO02/00900).

For the preparation of the expression vector MSP119, the 2.4 KB SacI-XhoI fragment from pJOBGEN:NP196 was ligated with the SacI-XhoI-cleaved vector pSUN3. MSP119 comprises fragment B-GENE the B-GENE promoter, fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

The preparation of an expression vector for the Agrobacterium-mediated transformation of the PDS-controlled NP196 ketolase from Nostoc punctiforme in Tagetes erecta was carried out using the binary vector pSUN5 (WO02/00900).

For the preparation of the expression vector MSP120, the 2.4 KB bp SacI-XhoI fragment from pJOBGEN:NP196 was ligated with the SacI-XhoI-cleaved vector pSUN5. MSP120 comprises fragment B-GENE the B-GENE promoter, fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

### EXAMPLE 6C

Preparation of Expression Vectors for the Flower-Specific Expression of the NP196 Ketolase from Nostoc punctiforme ATCC 29133 in Lycopersicon esculentum and Tagetes erecta

The expression of the NP196 ketolase from Nostoc punctiforme in Tagetes erecta was carried out with the transit peptide rbcS from pea (Anderson et al. 1986, Biochem J. 240:709-715). The expression was carried out under the control of the flower-specific promoter CHRC (chromoplast-specific carotenoid-associated protein) from Cucumis sativa (database entry AF099501).

The DNA fragment which comprises the CHRC promoter region (SEQ ID No. 106) from Lycopersicon esculentum was prepared by means of PCR using genomic DNA (isolated from Lycopersicon esculentum according to standard methods) and the primer CHRC-1 (SEQ ID No. 104) and CHRC-2 (SEQ ID No. 105).

The PCR conditions were as follows:

The PCR for the amplification of the DNA which comprises the CHRC promoter fragment was carried out in a 50 μl reaction batch, in which was comprised:

100 ng of genomic DNA from Lycopersicon esculentum

0.25 mM dNTPs

0.2 mM CHRC-1 (SEQ ID No. 101)

0.2 mM CHRC-2 (SEQ ID No. 102)

5 ul of 10×PCR buffer (Stratagene)

0.25 ul of Pfu polymerase (Stratagene)

28.8 ul of dist. water

The PCR was carried out under the following cycle conditions:

| | | |
|---|---|---|
| 1X | 94° C. | 2 minutes |
| 35X | 94° C. | 1 minute |
| | 50° C. | 1 minute |
| | 72° C. | 2 minute |
| 1X | 72° C. | 10 minutes |

The 1222 bp amplificate was cloned using standard methods in the PCR cloning vector pCR 2.1 (Invitrogen) and the plasmid pCHRC obtained.

The clone pB-GENE was therefore used for cloning in the expression vector pJOEPS:NP196 (described in Example 2).

The cloning was carried out by isolation of the 1540 bp SacI-HindIII fragment from pCHRC and ligation in the SacI-HindIII-cleaved vector pJOEPS:NP196. The clone which comprises the promoter CHRC instead of the original promoter EPSPS is called pJOCHRC:NP196. This expression cassette comprises the fragment NP196 in the correct orientation as the N-terminal fusion with the rbcS transit peptide.

The preparation of an expression vector for the Agrobacterium-mediated transformation of the PDS-controlled NP196 ketolase from Nostoc punctiforme in L. esculentum was carried out using the binary vector pSUN3 (WO02/00900).

For the preparation of the expression vector MSP121, the 2.6 KB SacI-XhoI fragment from pJOCHRC:NP196 was ligated with the SacI-XhoI-cleaved vector pSUN3. MSP121 comprises fragment CHRC the CHRC promoter, fragment rbcS TP FRAGMENT the rbcS transit peptide from pea (194 bp), fragment NP196 KETO CDS (761 bp), coding for the Nostoc punctiforme NP196 ketolase, fragment OCS terminator (192 bp) the polyadenylation signal of octopine synthase.

The preparation of an expression vector for the Agrobacterium-mediated transformation of the PDS-controlled NP196 ketolase from Nostoc punctiforme in Tagetes erecta was carried out using the binary vector pSUN5 (WO02/00900).

For the preparation of the expression vector MSP122, the 2.6 KB bp SacI-XhoI fragment from pJOCHRC:NP196 was ligated with the SacI-XhoI-cleaved vector pSUN5. MSP122 comprises fragment CHRC the CHRC promoter, fragment rbcS TP FRAGMENT the rbcS transit peptide from pea (194 bp), fragment NP196 KETO CDS. (761 bp), coding for the Nostoc punctiforme NP196 ketolase, fragment OCS terminator (192 bp) the polyadenylation signal of octopine synthase.

### EXAMPLE 7

Production of Transgenic Tagetes Plants

Tagetes seeds are sterilized and placed on germination medium (MS medium; Murashige and Skoog, Physiol. Plant. 15(1962), 473-497) pH 5.8, 2% sucrose). Germination is carried out in a temperature/light/time interval of 18 to 28° C./20-200 μE/3-16 weeks, but preferably at 21° C., 20 to 70 μE, for 4-8 weeks.

All leaves of the in vitro plants developed by then are harvested and cut transversely to the center rib. The leaf explants resulting thereby having a size of 10-60 mm² are stored in the course of the preparation in liquid MS medium at room temperature for at most 2 hours.

Any desired Agrobacterium tumefaciens strain, but preferably a supervirulent strain, such as, for example, EHAL 05 with an appropriate binary plasmid, which can comprise a selection marker gene (preferably bar or pat) and one or more trait or reporter genes (pS5FNR:NOST,pS5AP3:NOST pS5FNR:NP196, pS5EPS:NP196, pS5FNR:NP195, pS5EPS:NP195, pS5FNR:NODK and pS5EPS:NODK), is cultured overnight and used for the coculturing with the leaf material. The culturing of the bacterial strain can be carried out as follows: An individual colony of the appropriate strain is inoculated into YEB (0.1% yeast extract, 0.5% beef extract, 0.5% peptone, 0.5% sucrose, 0.5% magnesium sulfate×7H₂O) with 25 mg/l of kanamycin and cultured at 28° C. for 16 to 20 h. Subsequently, the bacterial suspension is harvested by centrifugation at 6000 g for 10 min and resuspended in liquid MS medium in such a way that an OD₆₀₀ of about 0.1 to 0.8 resulted. This suspension is used for the C-culturing with the leaf material.

Immediately before the coculturing, the MS medium in which the leaves have been stored is replaced by the bacterial suspension. The incubation of the leaves in the Agrobacteria suspension was carried out for 30 min with slight shaking at room temperature. Subsequently, the infected explants are placed on an agar (e.g. 0.8% plant agar (Duchefa, NL))-solidified MS medium comprising growth regulators, such as, for example, 3 mg/l of benzylaminopurine (BAP) and 1 mg/l of indolylacetic acid (IAA). The orientation of the leaves on the medium is insignificant. The culturing of the explants takes place for 1 to 8 days, but preferably for 6 days, in this connection the following conditions can be used: light intensity: 30-80 μmol/m²×sec, temperature: 22-24° C., light/dark change of 16/8 hours. Subsequently, the cocultured explants are transferred to fresh MS medium, preferably comprising the same growth regulators, this second medium additionally comprising an antibiotic for suppression of the bacterial growth. Timentin in a concentration of 200 to 500 mg/l is very suitable for this purpose. As a second selective component, one for the selection of the transformation result is employed. Phosphinothricin in a concentration of 1 to 5 mg/l selects very efficiently, but other selective components are also conceivable according to the process to be used.

After one to three weeks in each case, the transfer of the explants to fresh medium is carried out until sprout buds and small sprouts develop, which are then transferred for rooting to the same basal medium including timentin and PPT or alternative components with growth regulators, namely, for example, 0.5 mg/l of indolylbutyric acid (IBA) and 0.5 mg/l of gibberellic acid GA₃. Rooted sprouts can be transferred to the greenhouse.

In addition to the methods described, the following advantageous modifications are possible:

Before the explants are infected with the bacteria, they can be preincubated for 1 to 12 days, preferably 3-4, on the medium described above for the coculture. Subsequently, the infection, coculture and selective regeneration is carried out as described above.

The pH for the regeneration (normally 5.8) can be lowered to pH 5.2. The control of the growth of the Agrobacteria is thereby improved.

The addition of AgNO₃ (3-10 mg/l) to the regeneration medium improves the condition of the culture including the regeneration itself.

Components which reduce the phenol formation and are known to the person skilled in the art, such as, for example, citric acid, ascorbic acid, PVP and very many others, have a positive effect on the culture.

Liquid culture medium can also be used for the total process. The culture can also be incubated on commercially available carriers, which are positioned on the liquid medium.

According to the transformation method described above, the following lines were obtained with the following expression constructs:

With pS5FNR:NOST, for example, was obtained: MSP102-1, MSP102-2, MSP102-3,

With pS5AP3:NOST for example, was obtained: MSP104-1, MSP104-2, MSP104-3

With pS5FNR:NP196 was obtained: MSP106-1, MSP106-2, MSP106-3

With pS5EPS:NP196 was obtained: MSP108-1, MSP108-2, MSP108-3

With pS5FNR:NP195 was obtained: MSP110-1, MSP110-2, MSP110-3

With pS5EPS:NP195 was obtained: MSP112-1, MSP112-2, MSP112-3

With pS5FNR:NODK was obtained: MSP114-1, MSP114-2, MSP114-3

With pS5EPS:NODK was obtained: MSP116-1, MSP116-2, MSP116-3

With pS3PDS:NP196 was obtained: MSP117-1, MSP117-2, MSP117-3

With pS5PDS:NP196 was obtained: MSP118-1, MSP118-2, MSP118-3

With pS3CHRC:NP196 was obtained: MSP119-1, MSP119-2, MSP119-3

With pS5CHRC:NP196 was obtained: MSP120-1, MSP120-2, MSP120-3

With pS3BGEN:NP196 was obtained: MSP121-1, MSP121-2, MSP121-3

With pS5BGEN:NP196 was obtained: MSP122-1, MSP122-2, MSP122-3

### EXAMPLE 8

Enzymatic Lipase-Catalyzed Hydrolysis of Carotenoid Esters from Plant Material and Identification of the Carotenoids

General Working Procedure

a) Plant material ground in a mortar (e.g. petal material) (30-100 mg fresh weight) is extracted with 100% acetone (three times 500 μl; in each case shake for approximately 15 minutes). The solvent is evaporated. Carotenoids are subsequently taken up in 495 μl of acetone, 4.95 ml of potassium phosphate buffer (100 mM, pH7.4) are added and the solutions are well mixed. The addition of about 17 mg of bile salts (Sigma) and 149 μl of an NaCl/CaCl₂ solution (3M NaCl and 75 mM CaCl₂) is then carried out. The suspension is incubated for 30 minutes at 37° C. For the enzymatic hydrolysis of the carotenoid esters, 595 μl of a lipase solution (50 mg/ml of lipase Type 7 from Candida rugosa (Sigma)) are added and incubated with shaking at 37° C. After approximately 21 hours, an addition of 595 μl of lipase with fresh incubation of at least 5 hours at 37° C. was carried out again. Subsequently, approximately about 700 mg of Na₂SO₄ are dissolved in the solution. After addition of 1800 μl of petroleum ether, the carotenoids are extracted into the organic phase by vigorous mixing. This extraction by shaking is repeated until the organic phase remains colorless. The petroleum ether fractions are combined and the petroleum ether is evaporated. Free carotenoids are taken up in 100-120 μl of acetone. By means of HPLC and a C30 reverse-phase column, free carotenoids can be identified on the basis of retention time and UV-VIS spectra.

The bile salts or bile acid salts used are 1:1 mixtures of cholate and deoxycholate.

b) Working procedure for working up if only small amounts of carotenoid esters are present in the plant material

Alternatively, the hydrolysis of the carotenoid esters can be achieved by lipase from Candida rugosa after separation by means of thin layer chromatography. To this end, 50-100 mg of plant material are extracted three times with approximately 750 μl of acetone. The solvent extract is concentrated in vacuo in a rotrary evaporator (increased temperatures of 40-50° C. are tolerable). Addition of 300 μl of petroleum ether:acetone (ratio 5:1) and thorough mixing is then carried out. Suspended substances are sedimented by centrifugation (1-2 minutes). The upper phase is transferred to a new reaction vessel. The residue remaining is again extracted with 200 μl of petroleum ether: acetone (ratio 5:1) and suspended substances are removed by centrifugation. The two extracts are brought together (volume 500 μl) and the solvents are evaporated. The residue is resuspended in 30 μl of petroleum ether:acetone (ratio 5:1) and applied to a thin layer plate (silica gel 60, Merck). If more than one application is necessary for preparative-analytical purposes, several aliquots in each case having a fresh weight of 50-100 mg should be prepared in the manner described for the thin layer chromatographic separation.

The thin layer plate is developed in petroleum ether:acetone (ratio 5:1). Carotenoid bands can be identified visually on account of their color. Individual carotenoid bands are scraped off and can be pooled for preparative-analytical purposes. Using acetone, the carotenoids are eluted from the silica material; the solvent is evaporated in vacuo. For the hydrolysis of the carotenoid esters, the residue is dissolved in 495 μl of acetone, 17 mg of bile salts (Sigma), 4.95 ml of 0.1M potassium phosphate buffer (pH 7.4) and 149 μl (3M NaCl, 75 mM CaCl₂) are added. After thorough mixing, the solution is equilibrated for 30 min at 37° C. The addition of 595p of lipase of Candida rugosa (Sigma, stock solution of 50 mg/ml in 5 mM CaCl₂) is then carried out. Overnight, the incubation with lipase with shaking at 37° C. is carried out. After approximately 21 hours, the same amount of lipase is added again; the mixture is incubated again at 37° C. with shaking for at least 5 hours. The addition of 700 mg of Na₂SO₄ (anhydrous) is then carried out; the mixture is extracted by shaking with 1800 μl of petroleum ether for about 1 minute and the mixture is centrifuged at 3500 revolutions/minute for 5 minutes. The upper phase is transferred to a new reaction vessel and the extraction with shaking is repeated until the upper phase is colorless. The combined petroleum ether phase is concentrated in vacuo (temperatures of 40-50° C. are possible). The residue is dissolved in 120 μl of acetone, if necessary by means of ultrasound. The dissolved carotenoids can be separated by means of HPLC using a C30 column and quantified with the aid of reference substances.

### EXAMPLE 9

HPLC Analysis of Free Carotenoids

The analysis of the samples obtained according to the working procedures in Example 15 is carried out under the following conditions:

The following HPLC conditions were set.

Separating column: Prontosil C30 column, 250×4.6 mm, (Bischoff, Leonberg, Germany)

| | |
|---|---|
| Flow rate: | 1.0 ml/min |
| Eluents: | Eluent A - 100% methanol |
| | Eluent B - 80% methanol, 0.2% ammonium acetate |
| | Eluent C - 100% t-butyl methyl ether |
| Detection: | 300-530 nm |

Gradient profile:

| Time | Flow rate | % eluent A | % eluent B | % eluent C |
|---|---|---|---|---|
| 1.00 | 1.0 | 95.0 | 5.0 | 0 |
| 12.00 | 1.0 | 95.0 | 5.0 | 0 |
| 12.10 | 1.0 | 80.0 | 5.0 | 15.0 |
| 22.00 | 1.0 | 76.0 | 5.0 | 19.0 |
| 22.10 | 1.0 | 66.5 | 5.0 | 28.5 |
| 38.00 | 1.0 | 15.0 | 5.0 | 80.0 |
| 45.00 | 1.0 | 95.0 | 5.0 | 0 |
| 46.0 | 1.0 | 95.0 | 5.0 | 0 |

Some typical retention times for carotenoids formed according to the invention are, for example:

violaxanthin 11.7 min, astaxanthin 17.7 min, adonixanthin 19 min, adonirubin 19.9 min, zeaxanthin 21 min.

1-18. (canceled)

19. A method for expression of one or more gene in a plant of the genus Tagetes comprising utilizing one or more promoter selected from the group consisting of
A) EPSPS promoter,
B) B gene promoter,
C) PDS promoter, and
D) CHRC promoter,
with the proviso that genes from plants of the genus Tagetes which are expressed in wild-type plants of the genus Tagetes by the promoter are excluded.

20. The method according to claim 19, wherein expression takes place specifically in flowers.

21. The method according to claim 20, wherein expression takes place specifically in petals.

22. The method according to claim 19, wherein the EPSPS promoter comprises
1) a nucleic acid sequence of SEQ ID NO: 1, 2 or 3 or
2) a sequence derived from these sequences by substitution, insertion or deletion of nucleotides, which has an identity of at least 60% at nucleic acid level with the respective sequence of SEQ ID NO: 1, 2 or 3 or
3) a nucleic acid sequence which hybridizes with the nucleic acid sequence of SEQ ID NO: 1, 2 or 3 under stringent conditions or
4) functionally equivalent fragments of the sequences under 1), 2) or 3).

23. The method according to claim 19, wherein the B gene promoter comprises
1) a nucleic acid sequence of SEQ ID NO: 4, 5 or 6 or
2) a sequence derived from these sequences by substitution, insertion or deletion of nucleotides, which has an identity of at least 60% at nucleic acid level with the respective sequence of SEQ ID NO: 4, 5 or 6 or
3) a nucleic acid sequence which hybridizes with the nucleic acid sequence of SEQ ID NO: 4, 5 or 6 under stringent conditions or
4) functionally equivalent fragments of the sequences under 1), 2) or 3).

24. The method according to claim 19, wherein the PDS promoter comprises
1) a nucleic acid sequence of SEQ ID NO: 7, 8, 9 or 10 or
2) a sequence derived from these sequences by substitution, insertion or deletion of nucleotides, which has an identity of at least 60% at nucleic acid level with the respective sequence of SEQ ID NO: 7, 8, 9 or 10 or
3) a nucleic acid sequence which hybridizes with the nucleic acid sequence of SEQ ID NO: 7, 8, 9 or 10 under stringent conditions or
4) functionally equivalent fragments of the sequences under 1), 2) or 3).

25. The method according claim 19, wherein the CHRC promoter comprises
1) a nucleic acid sequence of SEQ ID NO: 11, 12, 13 or 14 or
2) a sequence derived from these sequences by substitution, insertion or deletion of nucleotides, which has an identity of at least 60% at nucleic acid level with the respective sequence of SEQ ID NO: 11, 12, 13 or 14 or
3) a nucleic acid sequence which hybridizes with the nucleic acid sequence of SEQ ID NO: 11, 12, 13 or 14 under stringent conditions or
4) functionally equivalent fragments of the sequences under 1), 2) or 3).

26. A genetically modified plant of the genus Tagetes, comprising a genetic modification leading to an increasing or causing of the expression rate of at least one gene in comparison with the wild-type plant and being due to regulation of expression of the gene in the plant by one or more promoter selected from the group consisting of EPSPS promoter, B gene promoter, PDS promoter, and CHRC promoter.

27. The genetically modified plant according to claim 26, wherein the regulation of the expression of a gene in the plant is achieved by means of one or more promoter selected from the group consisting of EPSPS promoter, B gene promoter, PDS promoter, and CHRC promoter, in that
a) the one or more promoter is inserted into the genome of the plant such that expression of one or more endogenous gene takes place under the control of the inserted promoter or
b) one or more gene is inserted into the genome of the plant such that expression of one or more of the inserted gene takes place under the control of an endogenous promoter selected from the group consisting of EPSPS promoter, B gene promoter, PDS promoter, and CHRC promoter or
c) one or more nucleic acid construct comprising at least one promoter selected from the group consisting of EPSPS promoter, B gene promoter, PDS promoter, and CHRC promoter and, functionally linked, one or more gene to be expressed is inserted into the plant.

28. A genetically modified plant of the genus Tagetes, comprising a promoter selected from the group consisting of EPSPS promoter, B gene promoter, PDS promoter, and CHRC promoter and, functionally linked, a gene to be expressed, with the proviso that genes from plants of the genus Tagetes, which are expressed in wild-type plants of the genus Tagetes by the respective promoter, are excluded.

29. The method according to claim 22, wherein expression takes place specifically in flowers.

30. The method according to claim 29, wherein expression takes place specifically in petals.

31. The method according to claim 23, wherein expression takes place specifically in flowers.

32. The method according to claim 31, wherein expression takes place specifically in petals.

33. The method according to claim 24, wherein expression takes place specifically in flowers.

34. The method according to claim 33, wherein expression takes place specifically in petals.

35. The method according to claim 25, wherein expression takes place specifically in flowers.

36. The method according to claim 35, wherein expression takes place specifically in petals.

37. The plant according to claim 26, wherein the EPSPS promoter comprises
1) a nucleic acid sequence of SEQ ID NO: 1, 2 or 3 or
2) a sequence derived from these sequences by substitution, insertion or deletion of nucleotides, which has an identity of at least 60% at nucleic acid level with the respective sequence of SEQ ID NO: 1, 2 or 3 or
3) a nucleic acid sequence which hybridizes with the nucleic acid sequence of SEQ ID NO: 1, 2 or 3 under stringent conditions or
4) functionally equivalent fragments of the sequences under 1), 2) or 3).

38. The plant according to claim 26, wherein the B gene promoter comprises
1) a nucleic acid sequence of SEQ ID NO: 4, 5 or 6 or
2) a sequence derived from these sequences by substitution, insertion or deletion of nucleotides, which has an identity of at least 60% at nucleic acid level with the respective sequence of SEQ ID NO: 4, 5 or 6 or
3) a nucleic acid sequence which hybridizes with the nucleic acid sequence of SEQ ID NO: 4, 5 or 6 under stringent conditions or
4) functionally equivalent fragments of the sequences under 1), 2) or 3).

39. The plant according to claim 26, wherein the PDS promoter comprises
1) a nucleic acid sequence of SEQ ID NO: 7, 8, 9 or 10 or
2) a sequence derived from these sequences by substitution, insertion or deletion of nucleotides, which has an identity of at least 60% at nucleic acid level with the respective sequence of SEQ ID NO: 7, 8, 9 or 10 or
3) a nucleic acid sequence which hybridizes with the nucleic acid sequence of SEQ ID NO: 7, 8, 9 or 10 under stringent conditions or
4) functionally equivalent fragments of the sequences under 1), 2) or 3).

40. The plant according claim 26, wherein the CHRC promoter comprises
1) a nucleic acid sequence of SEQ ID NO: 11, 12, 13 or 14 or
2) a sequence derived from these sequences by substitution, insertion or deletion of nucleotides, which has an identity of at least 60% at nucleic acid level with the respective sequence of SEQ ID NO: 11, 12, 13 or 14 or
3) a nucleic acid sequence which hybridizes with the nucleic acid sequence of SEQ ID NO: 11, 12, 13 or 14 under stringent conditions or
4) functionally equivalent fragments of the sequences under 1), 2) or 3).

41. The plant according to claim 28, wherein the EPSPS promoter comprises
1) a nucleic acid sequence of SEQ ID NO: 1, 2 or 3 or
2) a sequence derived from these sequences by substitution, insertion or deletion of nucleotides, which has an identity of at least 60% at nucleic acid level with the respective sequence of SEQ ID NO: 1, 2 or 3 or
3) a nucleic acid sequence which hybridizes with the nucleic acid sequence of SEQ ID NO: 1, 2 or 3 under stringent conditions or
4) functionally equivalent fragments of the sequences under 1), 2) or 3).

42. The plant according to claim 28, wherein the B gene promoter comprises
1) a nucleic acid sequence of SEQ ID NO: 4, 5 or 6 or
2) a sequence derived from these sequences by substitution, insertion or deletion of nucleotides, which has an identity of at least 60% at nucleic acid level with the respective sequence of SEQ ID NO: 4, 5 or 6 or
3) a nucleic acid sequence which hybridizes with the nucleic acid sequence of SEQ ID NO: 4, 5 or 6 under stringent conditions or
4) functionally equivalent fragments of the sequences under 1), 2) or 3).

43. The plant according to claim 28, wherein the PDS promoter comprises
1) a nucleic acid sequence of SEQ ID NO: 7, 8, 9 or 10 or
2) a sequence derived from these sequences by substitution, insertion or deletion of nucleotides, which has an identity of at least 60% at nucleic acid level with the respective sequence of SEQ ID NO: 7, 8, 9 or 10 or
3) a nucleic acid sequence which hybridizes with the nucleic acid sequence of SEQ ID NO: 7, 8, 9 or 10 under stringent conditions or
4) functionally equivalent fragments of the sequences under 1), 2) or 3).

44. The plant according claim 28, wherein the CHRC promoter comprises
1) a nucleic acid sequence of SEQ ID NO: 11, 12, 13 or 14 or
2) a sequence derived from these sequences by substitution, insertion or deletion of nucleotides, which has an identity of at least 60% at nucleic acid level with the respective sequence of SEQ ID NO: 11, 12, 13 or 14 or
3) a nucleic acid sequence which hybridizes with the nucleic acid sequence of SEQ ID NO: 11, 12, 13 or 14 under stringent conditions or
4) functionally equivalent fragments of the sequences under 1), 2) or 3).
